SEQUENCE LISTING (21 sequences)

SEQ ID NO: 1 - DNA, 1550 bp, synthetic construct
  misc_feature 1..1550: note = 1550F
  source 1..1550: mol_type = other DNA
ggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgaccgtttctcacctgccttgcgccaaggcagggggcgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcc

SEQ ID NO: 2 - DNA, 1034 bp, synthetic construct
  misc_feature 1..1034: note = CMV promoter
  source 1..1034: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagac

SEQ ID NO: 3 - protein, 627 aa, synthetic construct
  REGION 1..627: note = Antares2
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLWNGNKIIDERLINPDGSLLFRVTINGVTGWRLHERILARHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 4 - DNA, 367 bp, synthetic construct
  misc_feature 1..367: note = U6 promoter-gRNA-Rosa26-gRNA chimeric fragment
  source 1..367: mol_type = other DNA
caaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcgatgacgagatcacgcggttttagagctagaaatagcaagttaaaataaggctagtccgttatcaacttgaaaaagtggcaccgagtcggtgctttttt

SEQ ID NO: 5 - DNA, 367 bp, synthetic construct
  misc_feature 1..367: note = U6 promoter-gRNA-AAVS1-gRNA chimeric fragment
  source 1..367: mol_type = other DNA
caaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtccctagtggccccactgtgttttagagctagaaatagcaagttaaaataaggctagtccgttatcaacttgaaaaagtggcaccgagtcggtgctttttt

SEQ ID NO: 6 - DNA, 793 bp, synthetic construct
  misc_feature 1..793: note = 5'-homologous arm for Rosa26 locus
  source 1..793: mol_type = other DNA
aatggcgtattctggttggagtaaagctcctgtcagttacgccgtcgggagtacgcagccgcttagcgactctcgcgttgccccctgggtggggcgggtaggtaggtggggtgtagagatgctgggtgtgcgggcgcggccggcctcctgcggcgggaggggagggtcagtgaaatcggctctggcgcgggcgtcctcccaccctccccttccttcgggggagtcggtttacccgccgcctgcttgtcttcgacacctgattggctgtcgaagctgtgggaccgggcccttgctactggctcgagtctcacatgagcgaaaccactgcgcggggcgcgggggtggcggggaggcgggcgttggtacggtcctccccgaggccgagcgccgcagtgtctggccccgcgcccctgcgcaacgtggcaggaagcgcgcgctggaggcgggggcgggctgccggccgagacttctggatggcggcggccgcggctccgccccgggttcccaccgcctgaagggcgagacaagcccgacctgctacaggcactcgtgggggtgggggaggagcgggggtcggtccggctggtttgtgggtgggaggcgcttgttctccaaaaaccggcgcgagctgcaatcctgagggagctgcggtggaggaggtggagagaaggccgcacccttctgggcagggggaggggagtgccgcaatacctttatgggagttctctgctgcctcccgtcttgtaaggaccgccctgggcctggaagaagccctccctcctttcctcctcgc

SEQ ID NO: 7 - DNA, 457 bp, synthetic construct
  misc_feature 1..457: note = 3'-homologous arm for Rosa26 locus
  source 1..457: mol_type = other DNA
catgcctccatgtcgagtcgcttctcgattatgggcgggattcttttgcctaggcttaaggggctaacttggtccctgggcgttgccctgcaggggagtgagcagctgtaagatttgaggggcgactccgattagtttatcttcccacggactagagttggtgtcgaggttattgtaataagggtggggtagggaaatggagcttagtcattcacctggggctgattttatgcaacgagactgcggattatcactacttatcatttttggagcatttttctagagacagacataaagcatgatcacctgagttttataccatttgagacccttgctgcaccaccaaagtgtagcatcaggttaaatcttaatagaaaaattttagcttttgcttgagaaaccagtgcttccctccctcaccctctctccccaggctctctacccctttgcatcccta

SEQ ID NO: 8 - DNA, 797 bp, synthetic construct
  misc_feature 1..797: note = 5'-homologous arm for AAVS1 locus
  source 1..797: mol_type = other DNA
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccaca

SEQ ID NO: 9 - DNA, 845 bp, synthetic construct
  misc_feature 1..845: note = 3'-homologous arm for AAVS1 locus
  source 1..845: mol_type = other DNA
gtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 10 - protein, 728 aa, synthetic construct
  REGION 1..728: note = HGF-endogenous SP
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 11 - protein, 720 aa, synthetic construct
  REGION 1..720: note = HGF-TPA
MDAMKRGLCCVLLLCGAVFVSPSQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 12 - DNA, 7830 bp, synthetic construct
  misc_feature 1..7830: note = pKPBml-PNUC-EGFP
  source 1..7830: mol_type = other DNA
ctagtcgagtaaagcgcaaatcttttttaaccctagaaagatagtctgcgtaaaattgacgcatgcattcttgaaatatcgctctctctttctaaatagcgcgaatccgtcgctgtgcatttaggacatctcagtcgccgcttggagctcccgtgaggcgtgtttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactcatcacactggcggccgcggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgaccgtttctcacctgccttgcgccaaggcagggggcgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctccatcgatggtaccccggctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgactctagagcaactagtgggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaagcttaaatcccggcgcgccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccgctagcatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaagatcttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgccatatgtgataacttcgtatagcatacattatacgaagttatgaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcg

SEQ ID NO: 13 - DNA, 1192 bp, synthetic construct
  misc_feature 1..1192: note = EF1alpha
  source 1..1192: mol_type = other DNA
ctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggcca

SEQ ID NO: 14 - DNA, 1550 bp, synthetic construct
  misc_feature 1..1550: note = 1550R
  source 1..1550: mol_type = other DNA
ggagacgccgtggcccccgaagcagcgtgctttagaaagggaataagaattcccgcctccgcgccccactttcaccccagcggggcagcgtccgccatgtgaaagctccccatcccccacccccagtgaagggaaatggcgccgggaggctgagggtggggaagctgtttgtacgctcaggcctccgctcaagaccccgttcataaaccttaagccccactgctactgaattggtccgatttcctgcctctctcccacggaggcggctggccgacttccactgaggcgccaacggcctcgccatgcccttttcaataactcattgatttcaaacccgttacctccatcgcggactcagtcgcttcagcccgatttcccgcagccgagcgagatgagagagatctccgcggacgaacacgaaccggactcgtcctggcgctgtagtgagaactgccgctgctcgagaaacaactctgcgaggagcacctccgcacgggacccggcgctgctgctactgccgctagagccgctgccgccgcttttctagaaccttcccccccactaacgcgtcttccgctacgtcaggccgtcgcgtaaacgccctatccgccgccaatggcgggaaggctctacgccccaccttacgccaaatgcgtactcctcccacccttgcggccagagacagtacccgacgttacttccgtaaatgcgctcaatgaattgcggaaggctagagtcctgctagttactacctcttggaatagggtcccgccccctgccttggcgcaaggcaggtgagaaacggtcgcgcagtttgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggcc

SEQ ID NO: 15 - DNA, 1194 bp, synthetic construct
  misc_feature 1..1194: note = 1194F
  source 1..1194: mol_type = other DNA
ggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgaccgtttctcacctgccttgcgccaaggcagggggcgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgact

SEQ ID NO: 16 - DNA, 1194 bp, synthetic construct
  misc_feature 1..1194: note = 1194R
  source 1..1194: mol_type = other DNA
agtcgcttcagcccgatttcccgcagccgagcgagatgagagagatctccgcggacgaacacgaaccggactcgtcctggcgctgtagtgagaactgccgctgctcgagaaacaactctgcgaggagcacctccgcacgggacccggcgctgctgctactgccgctagagccgctgccgccgcttttctagaaccttcccccccactaacgcgtcttccgctacgtcaggccgtcgcgtaaacgccctatccgccgccaatggcgggaaggctctacgccccaccttacgccaaatgcgtactcctcccacccttgcggccagagacagtacccgacgttacttccgtaaatgcgctcaatgaattgcggaaggctagagtcctgctagttactacctcttggaatagggtcccgccccctgccttggcgcaaggcaggtgagaaacggtcgcgcagtttgaaattaacgccgacgggaggggcttaatccgcagcctggagatccagccccctcaacccgggaggtggtccctgcagttacgccaatgataacccccgccagaaaaatcttagtagccttccctttttgttttccgtgccccaactcggcggattgactcggccccttccggaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggcc

SEQ ID NO: 17 - DNA, 761 bp, synthetic construct
  misc_feature 1..761: note = SRF6-3F
  source 1..761: mol_type = other DNA
gcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaa

SEQ ID NO: 18 - DNA, 20 bp, synthetic construct
  misc_feature 1..20: note = pCAS-gRNA-Rosa26 targeting sequence
  source 1..20: mol_type = other DNA
ggcgatgacgagatcacgcg

SEQ ID NO: 19 - DNA, 20 bp, synthetic construct
  misc_feature 1..20: note = pCAS-gRNA-AAVS1 targeting sequence
  source 1..20: mol_type = other DNA
gtccctagtggccccactgt

SEQ ID NO: 20 - protein, 31 aa, synthetic construct
  REGION 1..31: note = Endogenous SP
MWVTKLLPALLLQHVLLHLLLLPIAIPYAEG

SEQ ID NO: 21 - protein, 697 aa, synthetic construct
  REGION 1..697: note = HGF
QRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

(19) United States
(12) Patent Application Publication
(10) Pub. No.: US 2026/0347794 A1
(43) Pub. Date: Nov. 5, 2026

(54) STEM CELLS MODIFIED WITH HEPATOCYTE GROWTH FACTOR, METHODS OF MAKING THE SAME AND METHODS OF USING THE SAME

(71) Applicant: NUWACELL BIOTECHNOLOGIES CO., LTD., Hefei, Anhui (CN)

(72) Inventors: Junying YU, Hefei, Anhui (CN); Hailan LI, Hefei, Anhui (CN); Wentao SHAO, Hefei, Anhui (CN); Luyan JIAO, Hefei, Anhui (CN); Ying ZHANG, Hefei, Anhui (CN)

(73) Assignee: NUWACELL BIOTECHNOLOGIES CO., LTD., Hefei, Anhui (CN)

(21) Appl. No.: 18/725,266

(22) PCT Filed: Dec. 1, 2023
(86) PCT No.: PCT/CN2023/135752

§ 371 Date: Jun. 28, 2024

(51) Int. Cl. C12N 5/074; A61K 35/545; A61P 11/00; C07K 14/475; C12N 15/90

(52) U.S. Cl. CPC C12N 5/0696; A61K 35/545; A61P 11/00; C07K 14/4753; C12N 15/907; C12N 2510/00

(57) ABSTRACT

Provided herein are genetically-modified cells such as pluripotent stem cells and mesenchymal stem cells and methods for producing the same. The gene editing strategy of the present disclosure achieves high expression of a transgene in the genetically modified cells.

### TECHNICAL FIELD

This disclosure belongs to the field of stem cell biology, and relates to genetically modified stem cells such as pluripotent stem cell (PSC) and induced mesenchymal stem cell (iMSC), methods for producing the same and applications thereof.

### INCORPORATION BY REFERENCE OF SEQUENCE LISTING

The Sequence Listing in an XML file, named as 42343WO_SequenceListing of 49,785 bytes, created on Nov. 17, 2023, is incorporated herein by reference.

### BACKGROUND

Mesenchymal stem cells (MSCs) are stem cells having multipotency and self-renewing potency. MSCs can differentiate into a variety of cells including osteoblasts, chondrocytes, and adipocytes. Furthermore, MSCs are known to have a paracrine effect and a cellular adhesive interaction by self-produced factors. On the basis of these effects, MSCs exert the capability of repairing and regenerating target tissues and cells as well as the capability of controlling an immune response, for example, in anti-inflammation, thereby providing a therapeutic effect on various diseases.

Various studies have shown the enormous benefits of using MSC cells that secrete various paracrine factors in therapies for treating diseases such as neurological disorders, inflammations, cardiac ischemia, diabetes, and bone and cartilage diseases. In particular, PSC-derived MSCs (iMSCs) are highly promising because PSCs represents a potentially unlimited source of therapeutically viable cells. However, there is a need to further improve the effectiveness and/or functional stability of iMSCs before a iMSC-based therapy becomes a viable therapy.

### SUMMARY

In accordance with the present disclosure, there is provided engineered PSC and iMSC which are modified with HGF at a target locus. The engineered stem cells as disclosed herein can highly express HGF to have an enhanced function in contrast to wild type stem cells. The present disclosure also provides cell populations, cell lines, and/or clonal cells of the engineered stem cells as disclosed herein.

In a first aspect, the present disclosure is directed to a genetically modified pluripotent stem cell (PSC), comprising an expression cassette integrated at a target locus in the genome thereof, wherein the expression cassette comprises an exogenous polynucleotide encoding a hepatocyte growth factor (HGF).

In some embodiments of this aspect, the target locus is Rosa26.

In some embodiments of this aspect, the expression cassette comprises a promoter operably linked to the polynucleotide encoding the HGF, wherein the promoter is selected from the group consisting of EF1a, PGK, CAG, CMV, CLP, and UBC.

In some embodiments of this aspect, the expression cassette further comprises an anti-silencing ubiquitous chromatin opening element (UCOE) such as 1550F shown in SEQ ID NO: 1, and the UCOE is operably linked to the promoter.

In some embodiments of this aspect, the promoter is the EF1a promoter.

In some embodiments of this aspect, the expression cassette comprises the combination of the EF1a promoter with the UCOE.

In some embodiments of this aspect, the expression cassette further comprises a polynucleotide coding a signal peptide for directing the extracellular secretion of the HGF.

In some embodiments of this aspect, the signal peptide is shown in SEQ ID NO: 20.

In a second aspect, the present disclosure is directed to a method of producing a genetically modified PSC, the method comprising: introducing into PSC a first construct comprising a site-specific endonuclease capable of introducing a double strand break at a target locus in the genome of the PSC and a second construct comprising an expression cassette comprising an exogenous polynucleotide encoding a hepatocyte growth factor (HGF) and a pair of homology arms specific to the target locus and flanking the expression cassette such that the expression cassette in the second construct is integrated into the genome of the PSC at the target locus via homologous recombination, thereby obtaining the genetically modified PSC.

In some embodiments of this aspect, the target locus is Rosa26.

In some embodiments of this aspect, the expression cassette comprises a promoter operably linked to the polynucleotide encoding the HGF, wherein the promoter is selected from the group consisting of EF1a, PGK, CAG, CMV, CLP, and UBC.

In some embodiments of this aspect, the expression cassette further comprises an anti-silencing ubiquitous chromatin opening element (UCOE) such as 1550F shown in SEQ ID NO: 1, and the UCOE is operably linked to the promoter.

In some embodiments of this aspect, the promoter is the EF1a promoter.

In some embodiments of this aspect, the expression cassette comprises the combination of the EF1a promoter with the UCOE.

In some embodiments of this aspect, the expression cassette further comprises a polynucleotide coding a signal peptide for directing the extracellular secretion of the HGF.

In some embodiments of this aspect, the signal peptide is shown in SEQ ID NO: 20.

In some embodiments of this aspect, the method further comprises continuously expanding the genetically modified PSC for multiple passages such as at least 4 or 9 passages.

In a third aspect, the present disclosure is directed to a genetically modified induced mesenchymal stem cell (iMSC), comprising an expression cassette integrated at a target locus in the genome thereof, wherein the expression cassette comprises an exogenous polynucleotide encoding a hepatocyte growth factor (HGF).

In some embodiments of this aspect, the target locus is Rosa26.

In some embodiments of this aspect, the expression cassette comprises a promoter operably linked to the polynucleotide encoding the HGF, wherein the promoter is selected from the group consisting of EF1a, PGK, CAG, CMV, CLP, and UBC.

In some embodiments of this aspect, the expression cassette further comprises an anti-silencing ubiquitous chromatin opening element (UCOE) such as 1550F shown in SEQ ID NO: 1, and the UCOE is operably linked to the promoter.

In some embodiments of this aspect, the promoter is the EF1a promoter.

In some embodiments of this aspect, the expression cassette comprises the combination of the EF1a promoter with the UCOE.

In some embodiments of this aspect, the expression cassette further comprises a polynucleotide coding a signal peptide for directing the extracellular secretion of the HGF.

In some embodiments of this aspect, the signal peptide is shown in SEQ ID NO: 20.

In some embodiments of this aspect, the genetically modified iMSC secretes the HGF at a level of at least 16 times as high as that of wild type iMSC.

In a fourth aspect, the present disclosure is directed to a method of producing a genetically modified iMSC, the method comprising: providing the genetically modified PSC as described herein; and differentiating the genetically modified PSC into iMSC, thereby producing the genetically modified iMSC.

In some embodiments of this aspect, the method further comprises continuously expanding the genetically modified iMSC for multiple passages such as at least 4 or 7 passages.

In a fifth aspect, the present disclosure is directed to a method of producing a genetically modified iMSC, the method comprising: introducing into iMSC a first construct comprising a site-specific endonuclease capable of introducing a double strand break at a target locus in the genome of the iMSC and a second construct comprising an expression cassette comprising an exogenous polynucleotide encoding a hepatocyte growth factor (HGF) and a pair of homology arms specific to the target locus and flanking the expression cassette such that the expression cassette in the second construct is integrated into the genome of the iMSC at the target locus via homologous recombination, thereby obtaining the genetically modified iMSC.

In some embodiments of this aspect, the target locus is Rosa26.

In some embodiments of this aspect, the expression cassette comprises a promoter operably linked to the polynucleotide encoding the HGF, wherein the promoter is selected from the group consisting of EF1a, PGK, CAG, CMV, CLP, and UBC.

In some embodiments of this aspect, the expression cassette further comprises an anti-silencing ubiquitous chromatin opening element (UCOE) such as 1550F shown in SEQ ID NO: 1, and the UCOE is operably linked to the promoter.

In some embodiments of this aspect, the promoter is the EF1a promoter.

In some embodiments of this aspect, the expression cassette comprises the combination of the EF1a promoter with the UCOE.

In some embodiments of this aspect, the expression cassette further comprises a polynucleotide coding a signal peptide for directing the extracellular secretion of the HGF.

In some embodiments of this aspect, the signal peptide is shown in SEQ ID NO: 20.

In some embodiments of this aspect, the method further comprises continuously expanding the genetically modified iMSC for multiple passages such as at least 4 or 7 passages.

In a sixth aspect, the present disclosure is directed to a pharmaceutical composition, comprising the genetically modified iMSC as disclosed herein and a pharmaceutically acceptable carrier.

In a seventh aspect, the present disclosure is directed to a use of the genetically modified iMSC as disclosed herein in the manufacture of a medicament for treating or preventing idiopathic pulmonary fibrosis (IPF).

### BRIEF DESCRIPTION OF THE DRAWINGS

FIGS. 1A-1D. A. A schematic diagram of knock-in of Antares2 at the AAVS1 locus and the Rosa26 locus of iPSC. The upper line shows the targeted genomic locus, and the lower line shows the donor vector where the cassette for insertion comprises the EF1a promoter, Antares2, and Neo (Neomycin resistance gene, linked by P2A), and it is flanked on either side by 5′ and 3′ homologous arms. B. Plasmid profile of the Cas-template vector for knock-in of Antares2 in the iPSC. C. Plasmid profile of the donor vector, pKI-Antares2, for knock-in of Antares2 in the iPSC. D. Flow cytometry analysis of Antares2 expression in AAVS1- and Rosa26 locus-knock-in iPSC cells. Wild type iPSC (WT-iPSC) was used as control.

FIGS. 2A-2E. A. Plasmid profile of an EGFP-expressing vector, pPBml-PNE-EGFP vector, for random integration of iPSCs and iMSCs with the EF1a promoter, which can be replaced with other promoters in plasmids used in B. B. Plasmid profile of the PBase-expressing vector for random integration of iPSCs and iMSCs with EGFP-expressing vectors driven by different promoters. C. Representative fluorescence microscopic images of EGFP expression in iMSCs and iPSCs at different days post transfection with piggy-bac vectors containing different promoters. D. Flow cytometry analysis of EGFP expression in iMSCs at different days post transfection with piggy-bac vectors containing different promoters. E. Flow cytometry analysis of EGFP expression in iPSCs at different days post transfection with piggy-bac vectors containing different promoters. In the figures, E represents EF1a promoter; UE represents UCOE-EF1a promoter; C represents CMV promoter; and UC represents UCOE-CMV promoter.

FIGS. 3A-3C. A. A schematic diagram of knock-in of Antares2 driven by E and UC promoters at the Rosa26 locus of iPSC. The upper line shows the targeted genomic locus, and the lower line shows the donor vector where the cassette for insertion comprises the EF1a promoter or UCOE-CMV promoter, Antares2 and Neo (Neomycin resistance gene, linked by P2A), and it is flanked on either side by 5′ and 3′ homologous arms. B. Expression levels of Antares2 in the engineered iPSCs of different passages (P1, P5 and P10) integrated with the E or UC promoter at the Rosa26 locus. Wild type iPSC (WT-iPSC) was used as control. C. Expression levels of Antares2 in the engineered iPSC-derived iMSCs of different passages (P0 and P4). WT-iPSC, UC-iPSC (iPSC-Rosa26-UC-Antares2) and E-iPSC (iPSC-Rosa26-E-Antares2) were used as controls.

FIGS. 4A-4C. A. Comparison of the effects of the exogenous TPA signal peptide and the endogenous signal peptide on the secretion of HGF protein in the transiently transfected iMSCs. B. A schematic diagram of knock-in of the EF1a promoter-HGF at the Rosa26 locus in iPSC. C. Flow cytometry analysis of HGF expression in WT-iPSC and the engineered E-HGF-iPSC.

FIGS. 5A-5E. A. Representative cell morphology on Day-1 (iPSCs), Day 0 (EB formation), P0 and P2 during iMSC derivation from the engineered E-HGF-iPSCs. B. Flow cytometry analysis of typical MSC surface markers on engineered E-HGF-iMSCs of P2. WT-iPSCs were used as control. C. Flow cytometry analysis of HGF expression in WT-iPSCs, WT-iMSCs and engineered E-HGF-iMSCs. D. HGF secretion from iMSCs derived from WT-iPSCs and engineered E-HGF-iPSCs at P1 and P10. E. HGF secretion from WT-iMSCs at P2 and P4 and engineered E-HGF-iMSCs at P2, P4 and P7.

FIGS. 6A-6E. A. Time line for WT-iMSC, E-HGF-iMSC or saline injection after bleomycin administration in C57BL/6N mice. B. Lung index analysis of mice in four different groups (n≥6). Error bars indicate standard deviation of different mice undergoing the same procedure. C. HYP contents of lung in four different groups (n≥6). Error bars indicate standard deviation of different mice undergoing the same procedure. D. H&E staining of lung sections in four different groups (n≥6). Scale bar: 200 μm. E. Fibrosis score of mice in four different groups analyzed by Masson staining (n≥6). Error bars indicate standard deviation of different mice undergoing the same procedure.

### DETAILED DESCRIPTION

Various objects and advantages of the reagents, compositions and methods as provided herein will become apparent from the following description taken in conjunction with the accompanying drawings wherein are set forth, by way of illustration and example, some embodiments of the present disclosure.

It is to be appreciated that some aspects, modes, embodiments, variations and features of the present disclosure are described below in various levels of detail in order to provide a substantial understanding of the present technology.

Reference throughout this specification to “first,” “second,” “third,” “fourth,” “fifth,” “sixth,” “seventh,” “eighth,” or “ninth” does not mean the order or sequence of the feature, structure (e.g., medium or composition) or characteristic described in connection with the reference and can be used only for the purpose of distinction.

Reference throughout this specification to “a first aspect,” “a second aspect,” “a third aspect,” “a fourth aspect,” “a fifth aspect,” “a sixth aspect,” “a seventh aspect,” “an eighth aspect,” or “a ninth aspect” means that a particular feature, structure or characteristic described in connection with the aspect is included in at least one or more aspects of the present disclosure. Also, the particular feature(s), structure(s), characteristic(s) or embodiment(s) in one aspect may be combined with those in one or more other aspects in any suitable manner.

Reference throughout this specification to “one embodiment,” “some embodiments,” “a preferred embodiment(s),” or “certain embodiments” means that a particular feature, structure or characteristic described in connection with the embodiment(s) is included in at least one or more embodiments of the present disclosure. Also, the particular feature(s), structure(s), or characteristic(s) in one embodiment may be combined with those in one or more other embodiments in any suitable manner.

It is to be understood that the present disclosure is not limited to particular uses, methods, reagents, compounds, compositions or biological systems, which can, of course, vary. It is also to be understood that the terminology used herein is for the purpose of describing particular embodiments only and is not intended to be limiting.

### DEFINITIONS

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

Unless otherwise specified, “a” or “an” means “one or more.”

As used herein, “about” means plus or minus 10%, or plus or minus 5%, or plus or minus 4%, or plus or minus 3%, or plus or minus 2%, or plus or minus 1%, as well as the specified number.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of). Further, in each instance herein any of the terms “comprising,” “consisting essentially of,” and “consisting of” may be replaced with either of the other two terms.

As used herein, the term “pluripotent stem cell” (PSC) refers to cells that have the capability to self-renew in an undifferentiated state and to differentiate into almost any cell type in the body. Pluripotent stem cells can be pluripotent and give rise during development to all derivatives of the three primary germ layers: ectoderm, endoderm and mesoderm. Pluripotent stem cells can be of human origin (e.g., human PSC or hPSC). Pluripotent stems cells can be induced pluripotent stem cells (iPSCs) or embryonic stem cells (ESCs). Pluripotent stems cells can also comprise naïve PSC cells (NPSCs) and extended pluripotent stem cells (EPSCs). In some embodiments, the pluripotent stem cells are human induced pluripotent stem cells (hiPSCs). ESCs (e.g., hESCs) and iPSCs (e.g., hiPSCs) are known in the art and can be readily obtained using conventional methods, for example, those described in the existing technologies, or commercially available products.

As used herein, the term “embryonic stem cells,” or “ESCs” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extraembryonic membranes or the placenta, i.e., are not totipotent. When used in the present disclosure, the embryonic stem cells or ESCs are sourced from commercially established human embryonic stem cell lines or human embryonic stem cells isolated or acquired from early embryos that have developed in vitro for not more than 14 days from fertilization.

As used herein, the term “induced pluripotent stem cells” or “iPSCs” means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature. Suitable methods for the generation of iPSCs from somatic or multipotent stem cells are well known to those of skill in the art. For example, iPSCs may be reliably generated from somatic cells by conventional reprogramming technologies. For example, a method for reprogramming erythrocyte progenitor cells to generate hiPSCs has been described in detail in CN108373998B, which is owned by the present applicant and the disclosure of which is incorporated herein by reference in its entirety.

As used herein, the term “pluripotency” or “pluripotent” refers to the developmental potential of a cell to differentiate into cells of all three germ layers (Ectoderm, mesoderm, and endoderm). Pluripotency can be determined, at least in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRAl-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

As used herein, the term “pluripotent stem cell morphology” refers to the classical morphological features of an embryonic stem cell. Normal embryonic stem cell morphology can be characterized as small and round in shape, with a high nucleus-to-cytoplasm ratio, the notable presence of nucleoli, and/or typical inter-cell spacing.

As used herein, the term “reprogramming” refers to a method of increasing the potency of a cell or dedifferentiating a cell to a less differentiated state. For example, a cell that has an increased cell potency can have more developmental plasticity (i.e., can differentiate into more cell types) compared to the same cell in the non-reprogrammed state. That is, a reprogrammed cell is one that is in a less differentiated state than the same cell in a non-reprogrammed state. “Reprogramming” can refer to de-differentiating a somatic cell, or a multipotent stem cell, into a pluripotent stem cell, also referred to as an induced pluripotent stem cell, or iPSC.

As used herein, the term “differentiation” refers to the process by which an unspecialized (“uncommitted”) or less specialized cell acquires the features of a specialized cell such as, for example, a blood cell or an immune cell. In some embodiments, a differentiated or differentiation-induced cell is one that has taken on a more specialized (“committed”) position within the lineage of a cell. For example, a human Pluripotent Stem Cell (hPSCs) can be differentiated into various more differentiated cell types, for example, a neural progenitor cell (e.g., midbrain dopaminergic progenitor), a mesenchymal stem cell (MSC), a hematopoietic progenitor cell, a lymphocyte, a cardiomyocyte, an immune cell, and other cell types, upon treatment with suitable differentiation factors in the cell culture medium. In some embodiments, the term “committed” is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type. The term “differentiation” herein is also referred to as “directed differentiation”.

As used herein, the term “genetically modified” or “genetic modification” means that a cell has been modified to comprise at least one exogenous gene in the genome thereof. In the context of this disclosure, the term “genetically modified” or “genetic modification” can be used interchangeably with “genetic engineering”, “genetically engineered”, “gene-edited” or “gene-editing”.

As used herein, the term “genetically-modified pluripotent stem cell” or “genetically-modified PSC” refers to pluripotent stem cell which has been modified to comprise at least one exogenous gene in the genome thereof.

As used herein, the term “mesenchymal stem cell” or “MSC” refers to a stem cell which can self-renew and also exhibit the capacity of trilineage differentiation into adipocytes, osteocytes, and chondrocytes. MSCs comprise primary MSCs and induced MSCs (also referred to as iMSCs). Examples of the primary MSCs include, for example, bone marrow-derived mesenchymal stem cells (BM-MSCs), placental-derived mesenchymal stem cells (P-MSCs), umbilical cord-derived mesenchymal stem cells (UC-MSCs), adipose-derived mesenchymal stem cells (A-MSCs), peripheral blood-derived mesenchymal stem cells (PB-MSCs) and dental pulp-derived mesenchymal stem cells (DP-MSCs).

As used herein, the term “induced mesenchymal stem cell” (iMSC) refers to a pluripotent stem cell-derived mesenchymal stem cell. iMSCs possess the morphological, structural (e.g., markers) and functional characteristics similar as the primary MSCs. For example, iMSCs have the potential to develop into adipocytes, osteocytes, and chondrocytes and express typical markers such as CD73, CD90 and CD105. The iMSCs can be derived from PSC of any source. In certain embodiments, the iMSCs are ESC-derived MSCs. In certain embodiments, the iMSCs are iPSC-derived MSCs. In certain embodiments, the iMSCs are NPSC-derived MSCs s. In certain embodiments, the iMSCs are EPSC-derived MSCs. In some embodiments, the iPSC is a human iPSC (hiPSC). There are several methods to produce iMSC from iPSC known in the art. For example, iMSC can be produced from iPSC according to the method disclosed in CN110592007B, which is incorporated herein by reference in its entirety. Briefly, the method comprises forming embryoid bodies from human pluripotent stem cells; differentiating the embryoid bodies into mesoderm cells; and differentiating the mesoderm cells into mesenchymal stem cells.

As used herein, the term “genetically-modified induced mesenchymal stem cell” or “genetically-modified iMSC” refers to induced mesenchymal stem cell which has been modified to comprise at least one exogenous gene in the genome thereof.

As used herein, the term “wild type iPSC” or “WT iPSC” refers to the iPSC which has been not genetically modified.

As used herein, the term “wild type iMSC” or “WT iMSC” refers to the iMSC which has not been genetically modified.

As used herein, the term “expression cassette” refers to the complete elements required to express a gene, including an operably linked promoter and gene coding sequence.

As used herein, the term “coding sequence” refers to that portion of a nucleic acid sequence which directly specifies the amino acid sequence of its protein product. The boundaries of the coding sequence are generally determined by the ribosome binding site (for prokaryotic cells) immediately upstream of the 5′ open reading frame of the mRNA and the transcription termination sequence immediately downstream of the 3′ open reading frame of the mRNA.

As used herein, the term “gene(s) of interest” or “polynucleotide(s) of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode a mRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof. In the present disclosure, the term when used in reference to sequence includes any sequence having at least 80%, at least 85%, at least 90%, at least 95% or at least 99% identity to the referred sequence.

As used herein, the term “hepatocyte growth factor” or “HGF”, also known as scatter factor (SF), refers to a paracrine cellular growth, motility and morphogenic factor. It is secreted by mesenchymal cells and targets and acts primarily upon epithelial cells and endothelial cells, but also acts on haemopoietic progenitor cells and T cells.

As used herein, the term “signal peptide” or “signal sequence” refers to a short peptide chain for directing the transport of a synthesized protein (e.g., HGF) to a secretory pathway, generally having a length of about 5 to 30 amino acids and located at N-terminal of the protein.

As used herein, the term “exogenous” is intended to mean that the referenced molecule or material or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell.

As used herein, the term “endogenous” refers to a referenced molecule or material or activity that is present in the host cell. Similarly, the term when used in reference to expression of an encoding nucleic acid refers to expression of an encoding nucleic acid contained within the cell and not exogenously introduced.

As used herein, the term “operably linked” or “operatively linked” refers to the association of nucleic acid sequences on a single nucleic acid fragment so that the function of one is affected by the other. For example, a promoter is operably linked with a coding sequence or functional DNA when it is capable of affecting the expression of that coding sequence or functional DNA (i.e., the coding sequence or functional DNA is under the transcriptional control of the promoter). Coding sequences can be operably linked to regulatory sequences in sense or antisense orientation.

By “targeted integration” or “integrated at a target locus” it is meant that the exogenous nucleotide(s) of a construct is inserted into the cell's chromosomal or mitochondrial DNA at a pre-selected site or integration site. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

By “random integration” it is meant that the exogenous nucleotide(s) of a construct is inserted into the cell's chromosomal or mitochondrial DNA at an arbitrary site or integration site.

By “stable transfection” it is meant that the foreign DNA is introduced into the genome of the transfected cell.

By “transient transfection” it is meant that the foreign DNA is introduced into a cell where the foreign DNA fails to integrate into the genome of the transfected cell. The non-integrated transgene is expressed for a period of time which is less than the period of time for expression of the gene if integrated into the genome.

As used herein, the term “overexpression” or “overexpress” refers to the expression of an expression product, such as a polypeptide or protein, at a level greater than the expression of the same expression product prior to a genetic modification of the host cell or in a comparable host which has not been genetically modified at defined conditions.

As used herein, the term “co-express” or “co-expression” refers to the concomitant or simultaneous expression of at least two or multiple polynucleotides (nucleic acid molecules, such as genes) in a host cell, cell line or cell culture at about the same or different amounts or ratios.

As used herein, the term “construct” refers to a macromolecule or complex of molecules comprising a polynucleotide to be delivered to a host cell, either in vitro or in vivo. A “vector” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

As used herein, the term “continuous expansion” or “continuously expanding” refers to the long-term expansion of cells where the cells are passaged for multiple passages. In the context of this disclosure, the continuous expansion can be used interchangeably with the prolonged expansion.

As used herein, the term “embryoid body” (EB) refers to a three-dimensional cluster that have been shown to mimic embryo development as it gives rise to numerous lineages within its three-dimensional area.

As used herein, the term “culture medium” refers to a culture medium which can support the survival, growth, propagation, maintenance and/or differentiation of cells in an in vitro environment. A culture medium may have a basal medium and one or more supplements.

As used herein, the term “differentiation culture medium” or “differentiation culture media” refers to a culture medium(s) which can support the differentiation of cells in an in vitro environment.

As used herein, the term “in vitro” refers generally to activities that take place outside an organism.

As used herein, the term “in vivo” refers generally to activities that take place inside an organism.

As used herein, the term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In some embodiments, such tissues or cells can be collected and frozen, and later thawed for ex vivo treatment. Tissue culture experiments or procedures lasting longer than a few days using living cells or tissue are typically considered to be “in vitro,” though in some embodiments this term can be used interchangeably with ex vivo.

As used herein, the term “cell population” or “population of cells” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “effective amount” refers to a quantity of an agent sufficient to achieve a beneficial or desired result upon administration. The amount of an agent administered to the subject can depend on the characteristics of the individual, such as general health, age, sex, body weight, effective concentration of the cells (e.g., iMSCs) administered, and tolerance to drugs. The skilled artisan will be able to determine appropriate dosages depending on these and other factors. An effective amount can be administered to a subject in one or more doses.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular medical intervention, or from whom cells are harvested). In some embodiments, the individual, patient or subject is a human.

As used herein, the terms “treatment,” “treat,” and “treating” refer to a clinical intervention aimed to reverse, alleviate, delay the onset of, or inhibit the progress, ameliorate, reduce severity of, prevent or delay the recurrence of a disease, disorder, and/or condition or one or more symptoms thereof, and/or improve one or more symptoms of a disease, disorder, and/or condition as described herein. Treatment, e.g., in the form of an iMSC or a population of iMSCs as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, treatment may be administered to a susceptible individual prior to the onset of symptoms (e.g., in light of genetic or other susceptibility factors). Treatment may also be continued after symptoms have resolved, for example to prevent or delay their recurrence. Treatment can result in improvement and/or resolution of one or more symptoms of a disease, disorder and/or condition.

As used herein, the terms “prevent,” “preventing,” and “prevention” refer to reducing the probability of developing a disease, disorder, or condition in a subject, who does not have, but is at risk of or susceptible to developing a disease, disorder, or condition.

### Genetically Modified Cells

In one aspect, there is provided a genetically modified pluripotent stem cell (PSC), comprising an expression cassette integrated at a target locus in the genome thereof, wherein the expression cassette comprises an exogenous polynucleotide encoding a hepatocyte growth factor (HGF). As compared to WT stem cells, the resulting engineered stem cells can overexpress HGF to have an enhanced function. Further, this gene editing strategy overcomes the current barrier in engineering primary MSCs from peripheral blood, umbilical cord blood or any other donor tissues, as such cells are limited in supply and difficult to engineer, with engineering of such cells often lacking reproducibility and uniformity.

In another aspect, there is provided a genetically modified induced mesenchymal stem cell (iMSC), comprising an expression cassette integrated at a target locus in the genome thereof, wherein the expression cassette comprises an exogenous polynucleotide encoding a hepatocyte growth factor (HGF).

Genome editing, or genomic editing, or genetic editing, which can be used interchangeably, is a type of genetic engineering in which DNA is inserted, deleted, and/or replaced in the genome of a targeted cell. Targeted genome editing (interchangeable with “targeted genomic editing” or “targeted genetic modification”) enables insertion, deletion, and/or substitution at pre-selected sites in the genome. When an endogenous sequence is inserted, deleted, and/or replaced at the pre-selected site during targeted editing, an endogenous gene comprising the affected sequence (e.g., Rosa26) can be knocked-out or knocked-down. Therefore, targeted editing may also be used to disrupt endogenous gene expression.

Similarly used herein is the term “targeted integration,” referring to a process involving insertion of one or more exogenous sequences, with or without deletion of an endogenous sequence at the insertion site. In comparison, random integration or gene editing (using transposon system, for example) are subject to position effects and silencing, producing their expression unreliable and unpredictable. In addition, random integration may activate protooncogene, which brings about safety issue.

### Gene Loci

Non-limiting examples of gene locus for targeted integration of HGF in this disclosure comprises Rosa26, AAVS1 and house-keeping gene locus, e,g, GAPDH. In some embodiments, the target locus comprises Rosa26, AAVS1 and/or a house-keeping gene locus. In some embodiments, the target locus comprises Rosa26, AAVS1 and/or GAPDH. In some embodiments, the target locus is Rosa26 and/or AAVS1. In some embodiments, the target locus is Rosa26.

ROSA26 is a locus used for constitutive, ubiquitous gene expression in mice. It was first isolated in a gene-trap mutagenesis screen of embryonic stem cells (ESCs) (see, e.g., Friedrich, G; Soriano, P (1991). “Promoter traps in embryonic stem cells: A genetic screen to identify and mutate developmental genes in mice”. Genes & Development. 5 (9): 1513-23). The human ROSA26 locus has been identified (Irion, Stefan; Luche, Hervé; Gadue, Paul; Fehling, Hans Joerg; Kennedy, Marion; Keller, Gordon (2007). “Identification and targeting of the ROSA26 locus in human embryonic stem cells”. Nature Biotechnology. 25 (12): 1477-82). ROSA stands for Reverse Orientation Splice Acceptor, named after the lentivirus genetrap vector. More details regarding ROSA26 can be found in NCBI database, which is incorporated herein by reference in its entirety. The target sequence of the Rosa26 locus can be chosen from Chr3: 9432781 . . . 9440914.

Adeno-associated virus integration site 1 (AAVS1) is a viral integration site that in humans is encoded by the AAVS1 gene located on chromosome 19 (see, e.g., Ward et al., Virology. 2012 Nov. 25; 433(2):356-66. doi: 10.1016/j.virol.2012.08.015. Epub 2012 Sep. 13; and Kotin et al., EMBO J. 1992 December; 11(13):5071-8. doi: 10.1002/j.1460-2075.1992.tb05614.x.). More details regarding AAVS1 locus can be found in NCBI database, which is incorporated herein by reference in its entirety. The target sequence of the AAVS1 locus can be chosen from GenBank: AC010327.8 (7774 . . . 11429).

In accordance with this disclosure, the locus for gene editing can affect the expression level of transgene such as HGF. It has been uniquely discovered herein that the targeted integration of HGF gene at Rosa 26 allows the production of engineered PSC and iMSC cells having significantly higher expression level for HGF (SEQ ID NO: 21) as compared with the integration at an other locus such as AAVS1. It is believed that the Rosa26 locus is more accessible to transcription access and less susceptible to epigenetic silencing in the stem cells. As described herein, the genetic modification at Rosa26 does not substantially influence the expression level of the HGF in both the PSC cells and final differentiated cells such as iMSCs.

In some embodiments, the expression cassette in the engineered PSC and iMSC comprises a promoter operably linked to the exogenous polynucleotide encoding the HGF. The promoter is a part of the gene, usually located upstream of the 5′ end of the structural gene, and is a DNA sequence that RNA polymerase recognizes, binds and initiates transcription. Any suitable promoter can be used in the genetically-modified PSCs and iMSCs described herein. Suitable promoters include, but are not limited to, cytomegalovirus (CMV) promoter. This promoter is a strong constitutive promoter capable of driving high-level expression of any polynucleotide sequence operably linked thereto. Another example of a suitable promoter is elongation growth factor-1α (EF-1α). However, other promoters can also be used, including but not limited to Ubiquitin C (UBC) promoter, Phosphoglycerate Kinase (PGK) promoter, CMV early enhancer/chicken beta actin (CAG) promoter, and CpG free promoter (CLP) promoter. Further, the use of any promoter or variant derived from the above promoters is also contemplated. The present disclosure includes modified nucleotide sequences obtained by the substitution, deletion and/or addition of one or more bases compared with the above promoter sequences, and the modification still retains the biological function of the promoter's high-efficiency expression in the engineered cells. In some embodiments, the present disclosure includes sequences having at least 95%, at least 97% or at least 99% sequence identity to any of the above promoter sequences, and possessing the biological function expressed in the engineered cells. In some embodiments, the promoter is selected from the group consisting of EF1a, PGK, CAG, CMV, CLP, and UBC. In some embodiments, the promoter is selected from an EF1a promoter, a CMV promoter, or CLP promoter.

Transgene in the engineered cells is susceptible to epigenetic silencing during the prolonged expansion of the cells. Among all the factors that affect the epigenetic silencing, the specific promoter used is of key importance. In some embodiments, the promoter is selected from an EF1a promoter. Among all the promoters tested, the EF1a promoter allows the highest and most stable HGF expression in the engineered cells over the prolonged expansion. Thus, the engineered stem cells can stably sustain the high expression of the HGF over the prolonged expansion thereof (e.g., expansion for at least 2, 4, 6, 8 or 10 passages), which makes it possible to continuously produce the consistent engineered stem cells suitable for clinical application at a large scale.

In some embodiments, transgene silencing during the prolonged expansion of the engineered stem cells can be rescued by introducing a Ubiquitous Chromatin Opening Element (UCOE) upstream of the promoter. Thus, in some embodiments, the expression cassette of the present disclosure further comprises a Ubiquitous Chromatin Opening Element (UCOE) operably linked to the promoter. In some embodiments, the expression cassette comprises the combination of the EF1a promoter with the UCOE.

In some embodiments, examples of the UCOE include, but are not limited to, 1550F (SEQ ID NO:1), 1550R (SEQ ID NO: 14), 1194F (SEQ ID NO: 15), 1194R (SEQ ID NO: 16), and SRF6-3F (SEQ ID NO: 17). In some embodiments, the UCOE comprises 1550F, 1550R and SRF6-3F. In some embodiments, the UCOE comprises 1550F.

In some embodiments, the expression cassette comprises a selectable marker gene. In some embodiments, the expression cassette does not comprise a selectable marker gene. Useful selectable marker gene includes, for example, antibiotic resistance genes such as Kanamycin (Kan), Neomycin (Neo), Tetracycline (Ter), Chloramphenicol (Cam), and the like.

When the expression cassette comprises a selectable marker gene, it can be linked to the exogenous polynucleotide via a linker. Examples of the linker include, but are not limited to IRES, F2A, E2A, P2A, and T2A.

The engineered stem cells as disclosed herein may further comprise one or more other gene modifications depending on the applications or purposes of the cells. In addition to the HGF, the engineered stem cells as disclosed herein can express one or more other exogenous proteins or peptides. In some embodiments, the other gene modifications comprise knock-out or knock-down of an additional endogenous gene. In some embodiments, the other gene modifications comprise knock-in of an additional exogenous gene. Examples of the additional exogenous gene which can be knocked-in comprise ACE2, IL21, GLP, IL22, and CTLA4 genes.

In some embodiments, the engineered stem cells as disclosed herein further comprise one or more additional exogenous polynucleotides of interest integrated at a target locus. The additional exogenous polynucleotide(s) of interest can be integrated at a target locus same as or different from that for the targeted integration of the HGF. In some embodiments, the engineered stem cells as disclosed herein further comprise one or more additional exogenous polynucleotides of interest integrated at Rosa26. In some embodiments, the engineered stem cells as disclosed herein further comprise one or more additional exogenous polynucleotides of interest integrated at one or more loci other than Rosa26. Examples of the loci other than Rosa26 comprise AAVS1, and a house-keeping gene locus, e,g, GAPDH.

The one or more exogenous polynucleotides of interest can be co-expressed. For example, the expression cassette described herein may comprise an exogenous polynucleotide encoding the HGF and one or more additional exogenous polynucleotides of interest. These exogenous polynucleotides of interest can be linked to each other by the linker. In some embodiments, the linker encodes a self-cleaving peptide. Examples of the linker include, but are not limited to an Internal Ribosome Entry Sequence (IRES) or 2A self-cleaving peptide. Examples of the 2A self-cleaving peptide include, but are not limited to F2A, E2A, P2A, and T2A.

In case where the expression cassette comprises two or more exogenous polynucleotides, all the exogenous polynucleotides can be driven by a common promoter or can be separately driven by different promoters. When the expression cassette comprises different promoters, different or same UCOE elements can be operably linked to these promoters, respectively.

In some embodiments, the expression cassette further comprises other regulatory sequences for gene expression. Examples of the regulatory sequences include, but are not limited to, enhancer, poly(A) tailing signal sequence, and the like.

Enhancer refers to a DNA sequence that increases the transcription frequency of genes linked to it, and enhancers increase the transcription of downstream genes through the promoter. Effective enhancers can be located at the 5′ end of the gene, or at the 3′ end of the gene, and some can also be located in the intron of the gene. The enhancer can increase the transcription frequency for gene. Examples of the enhancer include, but are not limited to, CMV enhancer, SV40 enhancer, HPV16 LCR enhancer, immunoglobulin heavy chain enhancer, HACNS1 enhancer, GADD45G enhancer, hormone responsive element (HRE), metal-regulated enhancer element (MRE).

In some embodiments, the expression cassette further comprises a polynucleotide coding a signal peptide (SP) for directing the extracellular secretion of the HGF. The signal peptide can be endogenous or exogenous. In some embodiments, the endogenous signal peptide is shown in SEQ ID NO: 20. In some embodiments, the exogenous signal peptide is selected from SIRP, C2, TPA, IFNG or TNF signal peptide. With the endogenous signal peptide, the engineered stem cells can exhibit an improved secretion level for the HGF in contrast to the counterparts with the exogenous signal peptide.

In some embodiments, the genetically modified iMSC secretes the HGF at a level of at least 16 times (e.g., 16, 18, 20, or 22 times) as high as that of wild type iMSC. In some embodiments, the genetically modified iMSC secretes the HGF at a level of at least 24 times (e.g., 24, 26, 28, or 30 times) as high as that of wild type iMSC.

### Methods for Producing Genetically Modified Cells

The present disclosure also relates to methods and compositions for producing the genetically-modified PSCs and iMSCs described herein.

In a further aspect, the present disclosure provides a method of producing a genetically modified PSC, the method comprising: introducing into PSC a first construct comprising a site-specific endonuclease capable of introducing a double strand break at a target locus in the genome of the PSC and a second construct comprising an expression cassette comprising an exogenous polynucleotide encoding a hepatocyte growth factor (HGF) and a pair of homology arms specific to the target locus and flanking the expression cassette such that the expression cassette in the second construct is integrated into the genome of the PSC at the target locus via homologous recombination, thereby obtaining the genetically modified PSC.

In a still further aspect, the present disclosure provides a method of producing a genetically modified iMSC, the method comprising: providing the genetically modified PSC described herein; and differentiating the genetically modified PSC into iMSC, thereby producing the genetically modified iMSC.

In a still another aspect, the present disclosure provides a method of producing a genetically modified iMSC, the method comprising: introducing into iMSC a first construct comprising a site-specific endonuclease capable of introducing a double strand break at a target locus in the genome of the iMSC and a second construct comprising an expression cassette comprising an exogenous polynucleotide encoding a hepatocyte growth factor (HGF) and a pair of homology arms specific to the target locus and flanking the expression cassette such that the expression cassette in the second construct is integrated into the genome of the iMSC at the target locus via homologous recombination, thereby obtaining the genetically modified iMSC.

In some embodiments, the target locus is Rosa26 and/or AAVS1. In some embodiments, the target locus is Rosa26.

Any PSC can be used in the present methods. PSCs include embryonic stem cells (ESCs), and/or induced pluripotent stem cells (iPSCs). It is preferable that the PSCs are iPSCs because iPSCs represent unlimited cell source for cell-based therapy. ESCs (e.g., hESCs) and iPSCs (e.g., hiPSCs) are known in the art and can be readily obtained using conventional methods, for example, those described in the existing technologies, or commercially available products. For example, CytoTune iPS 2.0 Sendai Reprogramming Kit (ThermoFisher Scientific) can be used to reliably generate induced pluripotent stem cells (iPSCs) from somatic cells, including PBMCs and T-cells. In some embodiments, hiPSCs are prepared according to the protocol described in CN108373998B, which is incorporated herein by reference in its entirety.

In the gene engineering, a vector is generally used to deliver a target gene into cells. In the present methods, a tool vector is used to introduce the first construct comprising a site-specific endonuclease capable of introducing the double strand break (DSB) and a donor vector is used to introduce the second construct comprising an expression cassette comprising the exogenous polynucleotide encoding the HGF. In certain embodiments, the constructs are vectors. Vectors as used herein generally include, but are not limited to, plasmids, bacteriophages, animal viruses, and cosmids. The vector may be an expression vector, including eukaryotic expression vectors, and viral expression vectors. The vector is preferably an eukaryotic expression vector. Viruses that can be used as vectors include, but are not limited to, retroviruses, adenoviruses, adeno-associated viruses, herpes viruses, and lentiviruses. In this disclosure, any suitable vectors, including well-known vectors, can be used to deliver the first and second constructs. The technology for constructing a recombination vector is common for a skilled artisan in the art of gene engineering.

In the donor vector, the expression cassette comprises a promoter operably linked to the polynucleotide encoding the HGF, wherein the promoter is selected from the group consisting of EF1a, PGK, CAG, CMV, CLP, and UBC. In some embodiments, the expression cassette further comprises an anti-silencing ubiquitous chromatin opening element (UCOE) such as 1550F shown in SEQ ID NO: 1, and the UCOE is operably linked to the promoter. In some embodiments, the promoter is the EF1a promoter. In some embodiments, the expression cassette comprises the combination of the EF1a promoter with the UCOE. In some embodiments, the expression cassette further comprises a polynucleotide coding a signal peptide for directing the extracellular secretion of the HGF. In some embodiments of this aspect, the signal peptide is shown in SEQ ID NO: 20. Other elements in the expression cassette of the donor vector have been described elsewhere, and the descriptions thereof are omitted herein for purpose of simplification.

As a tool for targeted integration described herein, available endonuclease capable of introducing a DSB include, but not limited to, zinc-finger nuclease (ZFN), transcription activator-like effector nuclease (TALEN), and CRISPR-Cas nuclease.

In some embodiments, the endonuclease capable of introducing a double strand break comprises ZFN. As known for a skilled in the art, ZFN is a targeted endonuclease having a nuclease fused to a zinc finger DNA binding domain. A zinc finger is a domain of about 30 amino acids within the zinc finger binding domain whose structure is stabilized through coordination of a zinc ion. Examples of zinc fingers include, but not limited to, C2H2zinc fingers, C3H zinc fingers, and C4zinc fingers. An example of a ZFN is a fusion polypeptide of the FokI nuclease domain with a zinc finger DNA binding domain.

In some embodiments, the endonuclease capable of introducing a double strand break comprises TALEN. TALEN is a targeted endonuclease having a nuclease fused to a TAL effector DNA binding domain. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). An example of a TALEN is a fusion polypeptide of the FokI nuclease domain with a TAL effector DNA binding domain.

In some embodiments, the endonuclease capable of introducing a double strand break comprises CRISPR-Cas nuclease. CRISPR/Cas system is a powerful technology used as gene editing tool to selectively modify DNA sequence at any specific location in the genome of a cell. The CRISPR-Cas systems have been categorized into two classes and six major types. An example of CRISPR/Cas system is CRISPR/Cas9 system. CRISPR-Cas9 system is based on nucleolytic activity of the endonuclease protein, Cas9, which is guided to the desired site in the genome by a specificity determinant RNA, termed as guide RNA (gRNA). Apart from these, another sequence known as protospacer adjacent motif (PAM), present adjacent to the target site, is recognized by the CRISPR/Cas9 system and is crucial for the functionality of Cas9. The Cas9 protein binds to the target location in the presence of gRNA, with high precision and performs a double strand break at the cleavage site. Using predesigned repair template, the knock-in of gene as intended can be achieved by Homology-directed Repair (HDR).

The technology for transfecting a vector such as donor vector or tool vector is common for a skilled artisan in the art. Examples of the method for transfection comprises electroporation, calcium phosphate coprecipitation, liposome transfection, gene gun bombardment and the like.

In some embodiments, the method for producing the genetically-modified PSC according to this disclosure further comprises expanding the genetically modified PSC. In some embodiments, this method further comprises continuously expanding the genetically modified PSC for multiple passages. As such, the consistent engineered PSC cells having stable high expression for the HGF can be continuously produced at a large scale.

In some embodiments, continuously expanding the genetically-modified PSC cell comprises expanding the genetically-modified PSC for at least 2, 3, or 4 passages. In some embodiments, continuously expanding the genetically-modified PSC comprises expanding the genetically-modified PSC for at least 5, 6, or 7 passages. In some embodiments, continuously expanding the genetically-modified PSC comprises expanding the genetically-modified PSCl for at least 8, 9, or 10 passages. The technology for expanding or continuously expanding the PSCs are conventional in the art. For example, the engineered PSCs can be cultured and expanded in a common expansion medium for wild type PSCs. Any suitable other expansion medium can be also used herein. Examples of the above expansion medium comprise E8 medium and ncEpic medium.

In some embodiments, the method for producing the genetically modified iMSC according to this disclosure further comprises expanding the genetically modified iMSC. In some embodiments, this method further comprises continuously expanding the genetically modified iMSC for multiple passages. As such, the consistent engineered iMSCs having stable high expression for the HGF can be continuously produced at a large scale.

In some embodiments, continuously expanding the genetically-modified iMSC comprises expanding the genetically-modified iMSC for at least 2, 3, or 4 passages. In some embodiments, continuously expanding the genetically-modified iMSC comprises expanding the genetically-modified iMSC for at least 5, 6, or 7 passages. The technology for expanding or continuously expanding the iMSCs are conventional in the art. For example, the engineered iMSCs can be cultured and expanded in a common expansion medium for wild type iMSCs or primary MSCs. Any suitable other expansion medium can be also used herein. Examples of the expansion medium comprise Mesencult-XF medium (Stem cell), StemPro MSC SFM Xeno-Free medium (Invirogen), MSCGM-CD medium (Lonza), and M5 medium (Nuwacell).

### Compositions

The present disclosure further provides cell populations or compositions comprising the genetically-modified PSC or iMSC as disclosed herein.

Also provided herein is a pharmaceutical composition comprising the genetically-modified iMSC as disclosed herein and a pharmaceutically acceptable carrier. The amount of cells used in the pharmaceutical composition that is effective in the treatment of a particular disorder or condition can depend on the nature of the disorder or condition and can be determined by standard clinical techniques. Pharmaceutical compositions, cell compositions or populations of the present disclosure can be administered before, during, and/or after the onset of the disease, disorder, and/or condition.

Pharmaceutically acceptable carriers are well known in the art. Exemplary pharmaceutically acceptable carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, and other solutes. Non-limiting examples of such pharmaceutically acceptable carriers comprise Multiple Electrolytes Injection, and Dextran injection.

### Use

Also provided herein is a use of the genetically-modified PSC or iMSC as disclosed herein in the manufacture of a medicament for treating or preventing diseases such as neurological disorders, cardiac ischemia, bone and cartilage diseases, idiopathic pulmonary fibrosis (IPF), inflammations such as endometrial injury or vascular diseases, diabetes or autoimmune diseases or the like.

In particular, the genetically modified iMSC as disclosed herein has improved efficacy for IPF. Thus, the present disclosure further provides a use of the genetically modified iMSC as disclosed herein in the manufacture of a medicament for treating or preventing IPF. The present disclosure also provides a method of treating or preventing IPF, comprising administrating any of the genetically modified iMSC or cell populations or pharmaceutical compositions thereof described herein to a subject in need thereof.

### General Methods

In practicing the present disclosure, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### EXAMPLES

### Materials

All reagents and apparatuses utilized throughout the Examples of the present disclosure are commercially available.

### Example 1: Optimal Selection of a Genomic Locus for iPSC Engineering by Targeted Integration

### Experimental Procedure:

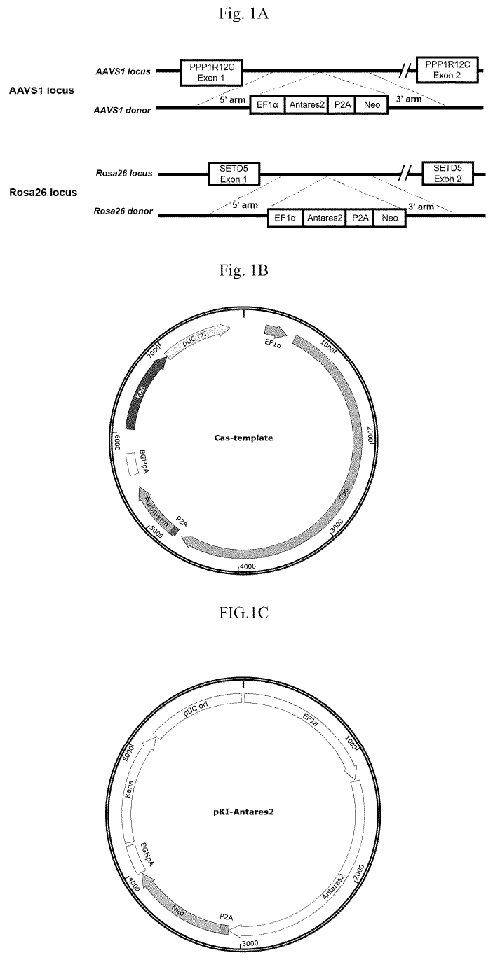

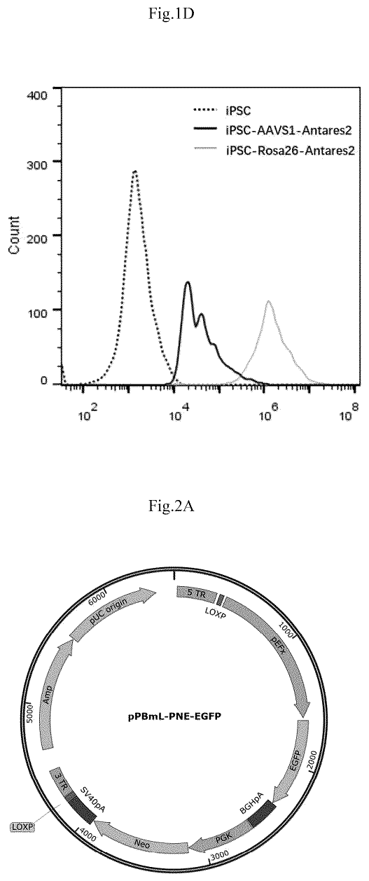

Human iPSCs (hiPSC cells) were prepared according to the protocol described in Examples 3 and 4 of CN108373998B. The hiPSCs were cultured and expanded in ncEpic medium (Nuwacell) for 4 days on a vitronectin-coated culturing surface. The hiPSCs were then engineered at the AAVS1 locus and the Rosa26 locus, respectively, to express Antares2. All procedures were the same except for targeting different loci. The following steps described the details to knock-in Antares2 (SEQ ID NO: 3) at the Rosa26 locus and the AAVS1 locus (FIG. 1A).

U6 promoter-gRNA-Rosa26-gRNA chimeric fragment (SEQ ID NO: 4) and U6 promoter-gRNA-AAVS1-gRNA chimeric fragment (SEQ ID NO: 5) were synthesized by GenScript Inc. (China). The synthesized U6 promoter-gRNA-Rosa26-gRNA chimeric fragment or U6 promoter-gRNA-AAVS1-gRNA chimeric fragment was inserted into KpnI/EcoRI site of a Cas-Template vector (FIG. 1B, Nuwacell) by carrying out double digestion at 37 °C for 1-2 hours, extracting the products with DNA Gel Extraction Kit (TIANGEN, DP209) according to manufacturer's instructions, and ligating the products with T4 ligase (NEB, M0202) according to manufacturer's instructions, thereby constructing a Cas/gRNA vector. The Cas/gRNA vectors and the gRNA sequences used are shown in Table 1 below.

TABLE 1

| Cas/gRNA Vector | Construct | Chr# | Exon# | Targeting sequence | PAM |
|---|---|---|---|---|---|
| pCas-gRNA-Rosa26 | gRNA-Rosa26 | 3 | N/A | GGCGATGACGA | AGG |
| | | | | GATCACGCG | |
| | | | | (SEQ ID NO: 18) | |
| pCas-gRNA-AAVS1 | gRNA-AAVS1 | 19 | N/A | GTCCCTAGTGGC | GGG |
| | | | | CCCACTGT (SEQ | |
| | | | | ID NO: 19) | |

5′- and 3′-homologous arms were synthesized by GenScript Inc. (China). The synthesized homologous arms were inserted into NheI/ClaI site and EcoRI/BamHI site of a pKI-Antares2 vector (FIG. 1C, Nuwacell), respectively, by carrying out double digestion at 37 °C for 1-2 hours, extracting the products with DNA Gel Extraction Kit (TIANGEN, DP209) according to manufacturer's instructions, and ligating the products with T4 ligase (NEB, M0202) according to manufacturer's instructions, thereby constructing a donor vector. The homologous arms and the donor vector for each locus are shown in Table 2 below.

TABLE 2

| | 5′-homologous | 3′- homologous | |
|---|---|---|---|
| Donor vector | arm | arm | Target locus |
| pKI-Antares2-Rosa26 | SEQ ID NO: 6 | SEQ ID NO: 7 | Rosa26 |
| pKI-Antares2-AAVS1 | SEQ ID NO: 8 | SEQ ID NO: 9 | AAVS1 |

To knock-in Antares2 in iPSCs, 2×10⁶ hiPSCs were transfected with 2 μg of the donor vector and 2 μg of Cas/gRNA vector by Nucleofector 2b (Lonza Inc.). The transfected hiPSC cells were plated at a cell density of 2×10⁴ cells/cm² in six well plates and selected with 100 ng/mL geneticin for 1 to 2 days. After growing for 5 to 7 days, hiPSC single clones were picked and transferred into 48 well plates, and then further expanded in ncEpic medium (Nuwacell) into 6 well plates to get enough cells for further screening. The hiPSCs from single clones were collected separately and dissociated into single cells, and the expression of Antares2 was analyzed by flow cytometry assay.

Positive clones where the Antares2 sequence was correctly inserted at the chosen locus of the genome were further confirmed by nested PCR assay using the Platinum® Pfx DNA Polymerase (ThermoFisher Scientific) according to the manual instruction and subjected to sequencing by Tsingke Biotechnology Co., Ltd. (China).

The positive clones with correct insertion were then karyotyped by KingMed Diagnostics Inc. (China) to exclude chromosomal abnormalities. Expression of Antares2 in representative double knock-in hiPSCs was analyzed by flow cytometry assay (FIG. 1D).

In the above assay, the wild type iPSC (hereinafter referred to as WT-iPSC) was used as control. As shown in FIG. 1D, the expression level of Antares2 in the engineered iPSC integrated at the Rosa26 locus or the AAVS1 locus was greatly higher than that of the WT-iPSC. Further, the expression level of Antares2 integrated at the Rosa26 locus was significantly superior to the AAVS1 locus, suggesting that the Rosa26 locus is more accessible to transcription access in iPSC cells. The above example demonstrated that the targeted integration in iPSC allowed high-level expression of HGF, and the Rosa26 locus outperformed the AAVS1 locus for iPSC engineering and subsequent iMSC engineering.

### Example 2: Pre-Selection of the Promoter for iPSC and iMSC Engineering by Random Integration

### Experimental Procedure:

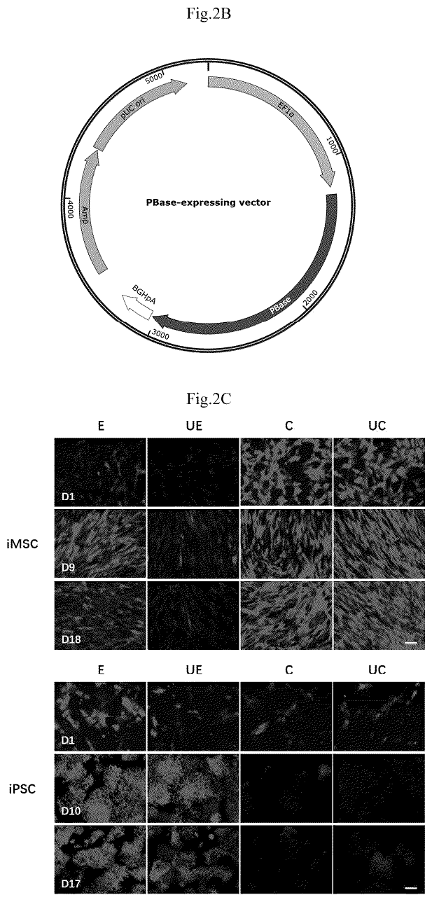

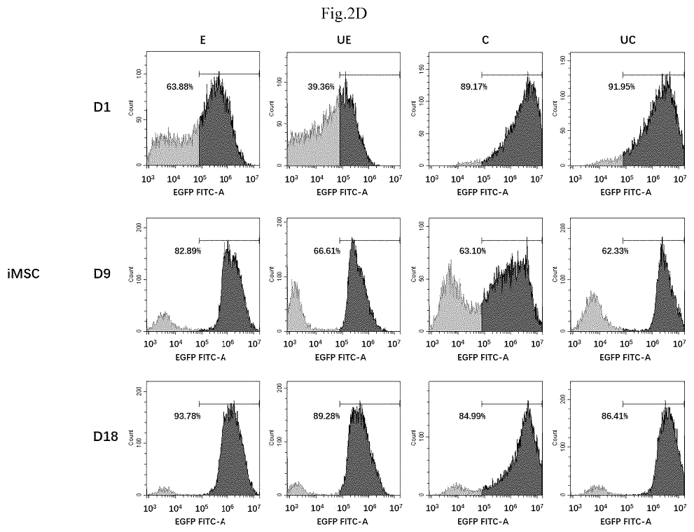

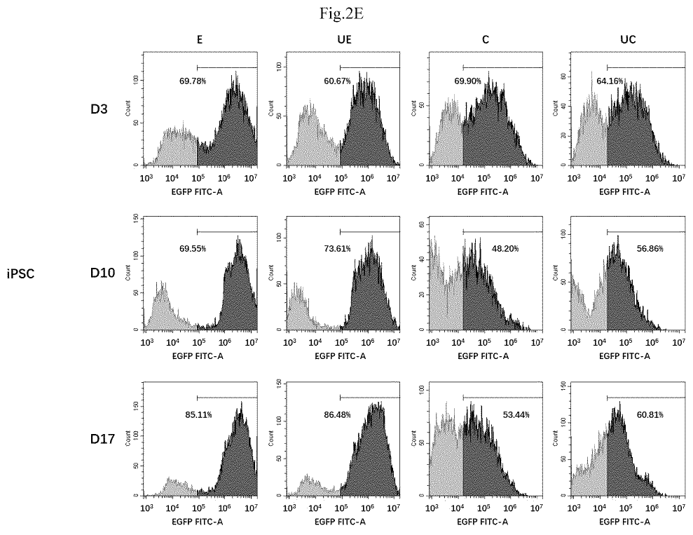

pPBml-PNE-EGFP vector (FIG. 2A, Nuwacell) was used as an EGFP-expressing vector. The vector had an EF1a promoter (SEQ ID NO: 13). A 1550F UCOE element (SEQ ID NO:1) and a CMV promoter (SEQ ID NO:2) were synthesized by GenScript Inc. (China). The 1550F UCOE element was inserted into NotI/ClaI site of the pPBml-PNE-EGFP vector by T4 ligase (NEB M0202) according to instructions. The CMV promoter was ligated into a ClaI/XbaI site of the pPBml-PNE-EGFP vector.

hiPSCs were prepared according to the protocol described in Examples 3 and 4 of CN108373998B. hiPSCs were cultured and expanded in ncEpic medium (Nuwacell) for 4 days on a vitronectin-coated surface. For hiPSC transfection, hiPSCs were dissociated by TrypLE treatment, and 2×10⁶ hiPSCs were used for each nucleofection (Nucleofector 2b, Lonza Inc.) with 2 μg of a PBase-expressing vector (FIG. 2B, Nuwacell) and 2 μg of the EGFP-expressing vector. Geneticin selection (100 pg/mL) was started on day 1 post-transfection for stable vector integration.

iMSCs were differentiated from hiPSCs according to the protocol described in Examples 1, 3 and 4 of patent CN110592007B. The iMSCs were cultured and expanded in an expansion medium (Nuwacell, Product Name: M5). For iMSC transfection, the iMSC cells were dissociated with TrypLE treatment, and 1×10⁶ iMSCs were used for each nucleofection with 2 μg of the PBase-expressing vector and 2 μg of the EGFP-expressing vector. Geneticin selection (100 μg/mL) was started on day 1 after transfection for stable vector integration. The EGFP expressions in the hiPSCs and iMSCs were visualized by fluorescence microscopy (FIG. 2C) and measured by flow cytometry (FIGS. 2D and 2E) on different days after transfection.

To find the optimal promoter for high-level transgene expression in iMSCs over prolonged culture, the EF1α (E) promoter, the CMV (C) promoter, and their combination with the anti-silencing ubiquitous chromatin opening elements (UCOE) (UCOE-EF1a (UE) and UCOE-CMV (UC)) were tested as above. Taking the results of FIGS. 2C-2E together, it was found that the EGFP expression in the iMSCs was more uniform and higher with the E promoter over prolonged culture than the UE promoter, while the EGFP expression in the iMSCs was more uniform and stable with the UC promoter over prolonged culture than the C promoter. Different from iMSCs, the EGFP expression in iPSC cells was higher with the E and UE promoters over prolonged culture, and although the iPSCs showed good expression of EGFP with the C and UC promoters on Day 1, the EGFP was efficiently silenced over prolonged culture. As potential effective drivers for transgene expression in the iMSCs over prolonged culture, the E and UC promoters were selected for further test in iPSC and iMSC engineering at the Rosa26 locus.

### Example 3: Comparison of the E and UC Promoter Activity During the Prolonged Expansion of Engineered iPSC Cells and iMSC Cells with Targeted Integration at the Rosa26 Locus

### Experimental Procedure:

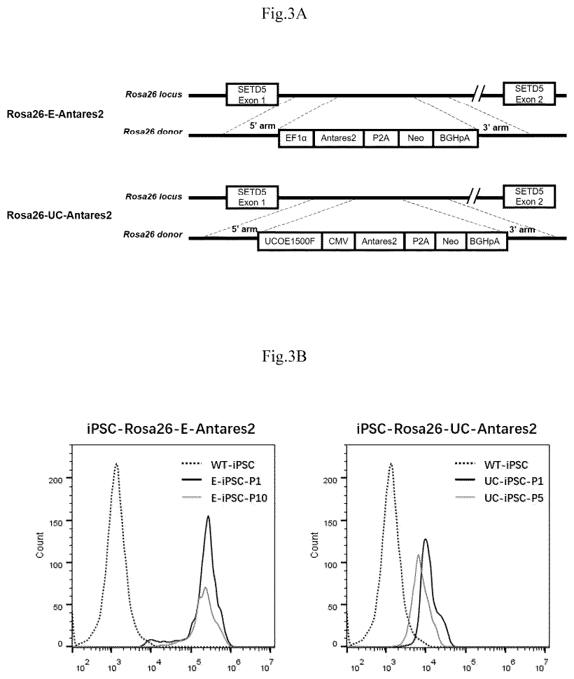

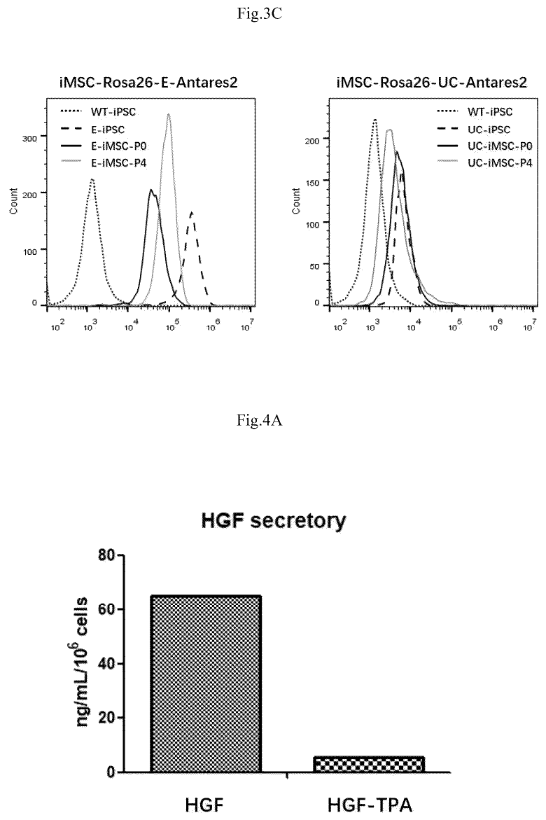

hiPSCs were prepared according to the protocol described in Examples 3 and 4 of CN108373998B. The hiPSCs were cultured and expanded in ncEpic medium (Nuwacell) for 4 days. Antares2 driven by the EF1a and UCOE-CMV promoters were knocked-in at the Rosa26 locus in the hiPSCs similarly to the methods described in Example 1. All procedures were the same except for the promoter (FIG. 3A). The hiPSCs engineered with the EF1a and UCOE-CMV promoters were named as iPSC-Rosa26-E-Antares2 and iPSC-Rosa26-UC-Antares2, respectively. The first passage of confirmed engineered iPSCs was referred to as “P1” (Passage 1). The iPSC-Rosa26-E-Antares2 cells of P1 were expanded and passaged in ncEpic medium (Nuwacell) for 9_passages, and the iPSC-Rosa26-UC-Antares2 cells of P1 were expanded and passaged in ncEpic medium (Nuwacell) for 4 passages. The Antares2 expression in the engineered iPSCs was analyzed by flow cytometry (FIG. 3B).

In the above assay, the WT iPSC was used as control. As shown in FIG. 3B, the expression of Antares2 in the iPSC-Rosa26-E-Antares2 cells was very high and could be stably maintained even after the hiPSCs had been expanded and passaged for 9 passages, and although the iPSC-Rosa26-UC-Antares2 cells showed good expression of Antares2, but it was reduced during prolonged expansion.

The iPSC-Rosa26-E-Antares2 cells of P1 and the iPSC-Rosa26-UC-Antares2 cells of P1 were differentiated according to the protocol described in Examples 1, 3 and 4 of patent CN110592007B to obtain iMSCs, and then the iMSCs were cultured and expanded in an expansion medium (Nuwacell, Product Name: M5) for 4 passages. The Antares2 expression in the iMSCs was analyzed by flow cytometry at P0 and P4 (FIG. 3C).

As shown in FIG. 3C, the iMSC-Rosa26-E-Antares2 cells showed high expression of Antares2 at P0, and increased expression at P4, while the iMSC-Rosa26-UC-Antares2 cells showed lower expression of Antares2 at P0 than the iMSC-Rosa26-E-Antares2 cells, and even weaker expression at P4.

Here, the results showed that transgene driven by the EF1a promoter integrated at the Rosa26 locus allowed the production of iPSCs with stable and high-level expression of transgene during the prolonged expansion and the production of iMSCs with stable and high-level expression of transgene during both the differentiation and the prolonged expansion. With the UC promoter, despite its ability to drive high transgene expression when directly introduced into iMSCs, it failed to be reactivated to its full strength during the prolonged expansion of iPSCs and/or iMSCs, which resulted in the silencing of the transgene.

### Examples 4-5: Generation of Engineered E-HGF-iPSC Cells by Targeted Integration at Rosa26

### Example 4

### Experimental Procedure:

hiPSCs were prepared by the protocol described in Examples 3 and 4 of CN108373998B, and differentiated into iMSCs by the protocol described in Examples 1, 3 and 4 of CN110592007B. The nucleic acid sequences of HGF (with endogenous SP) (SEQ ID NO: 10) and HGF-TPA (SEQ ID NO: 11) were synthesized by the GenScript Inc. (China). The above HGF and HGF-TPA sequences were inserted into a NotI/BamHI site of the pKPBml-PNUC-EGFP vector (SEQ ID NO:12) (Nuwacell), respectively, by T4 ligase (NEB M0202) according to manual instructions in order to construct pKPBml-PNUC-HGF and pKPBml-PNUC-HGF-TFA vectors, and the iMSC cells were transfected with these two constructs, respectively. The transfected iMSCs were cultured in an expansion medium (Nuwacell, Product Name: M5) for 4 days, and then the culture supernatants were collected to determine the HGF protein concentration by ELISA.

### Enzyme-Linked Immunosorbent Assay (ELISA):

To analyze the amount of HGF expressed by the iMSCs, culture media were collected and analyzed by ELISA at 4 days after culture. The levels of HGF were quantified using a Human HGF ELISA Kit (Multisciences), according to manufacturer's instructions. After applying STOP solution (Multisciences) to determine the reaction, the absorbance was measured at 450 nm.

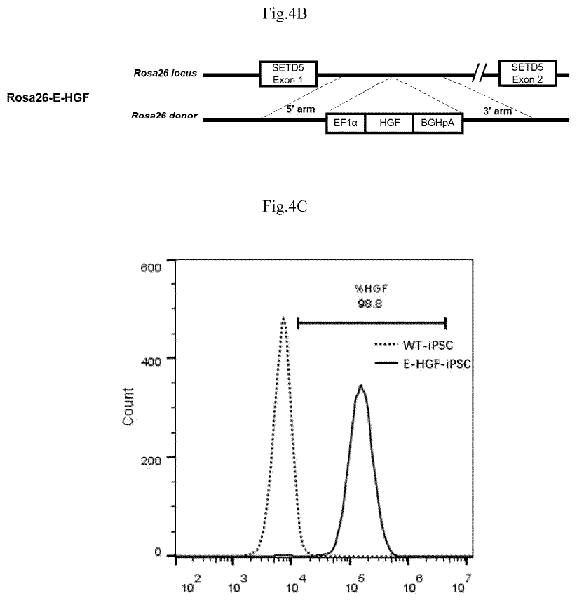

As the preliminary experiment, the effect of the TPA signal peptide on the secretion of HGF in the transiently transfected iMSCs was tested as above. As shown in FIG. 4A, as compared to the endogenous signal peptide, the exogenous TPA signal peptide resulted in the reduction of HGF protein secretion when introduced into the iMSCs. Thus, the exogenous TPA signal peptide was not considered with priority in subsequent gene engineering with targeted integration.

### Example 5

### Experimental Procedure:

hiPSCs were prepared according to the protocol described in Examples 3 and 4 of CN108373998B. The hiPSCs were cultured and expanded in ncEpic medium (Nuwacell) for 4 days. HGF (SEQ ID NO: 10) driven by the EF1aα promoter was knocked-in at the Rosa26 locus in the hiPSCs in a method similar to that described in Example 1 (FIG. 4B). The engineered iPSCs were named as E-HGF-iPSC cells. The confirmed E-HGF-iPSCs cand the WT iPSCs were stained with an anti-HGF Antibody (Sino Biological, 10463-T26), and the percentage of HGF+ iPSCs was detected by flow cytometry (FIG. 4C). The process for HGF staining was as follows. Cultured iPSCs were subjected to a TrypLE treatment. The collected cells were washed with 1 ml PBS and then centrifugated at 300×g for 15 sec. After removing the supernatant, ~200 μl of 4% PFA was added and mixed with the cells, and the cells were fixed for 10 min at RT. The cells were then centrifugated at 350×g for 5 min. The cells were then washed with 1 ml of an FACS buffer and centrifugated at 350×g for 5 min. After removing the supernatant, 200 μl/tube of the FACS buffer/0.1% Triton X was added and mixed with the cells, and the mixture was incubated for 10 min at RT. The cells were washed again with 1 ml of the FACS buffer and centrifugated at 350×g for 5 min. After removing the supernatant, 200 μl/tube of the anti-HGF antibody (Rabbit polyclonal, Sino Biological, #10463-T6) diluted in FACS buffer was added and mixed gently with the cells, and the mixture was incubated for 30 min at RT. The cells were washed again in 1 ml of the FACS buffer and centrifugated at 350×g for 5 min. After removing the supernatant, 200 μl of a goat anti-rabbit IgG H&L (APC) diluted in the FACS buffer was added and mixed gently with the cells, and the mixture was incubated for 30 min at RT. The cells were washed using 1 ml of the FACS buffer and centrifugated at 350×g for 5 min. After removing the supernatant, 200 μl of the FACS buffer was added, and the samples were run on the flow cytometer.

As shown in FIG. 4C, almost all the iPSCs expressed the HGF. The above results showed that the targeted integration of HGF by the EF1a promoter at the Rosa26 locus in iPSCs allowed the production of engineered iPSCs with high purity.

### Examples 6-7: Generation of Engineered E-HGF-iMSCs by Targeted Integration at Rosa26

### Example 6

### Experimental Procedure:

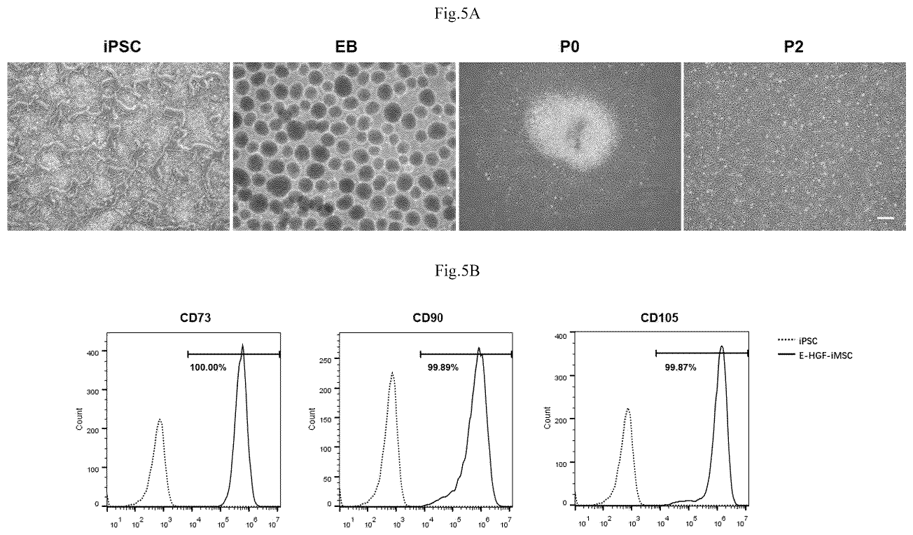

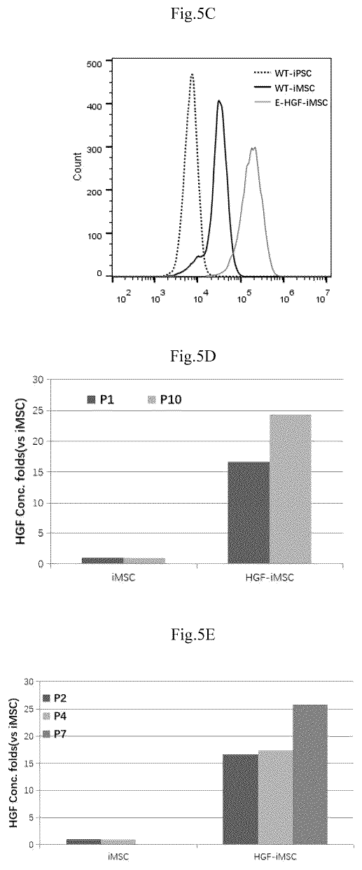

The engineered iPSCs (E-HGF-iPSCs) produced in Example 5 were differentiated for 10 days according to the protocol described in Examples 1, 3 and 4 of Patent CN110592007B to obtain engineered iMSCs (hereinafter referred to as E-HGF-iMSCs) and the E-HGF-iMSCs were then expanded and passaged in an expansion medium (Nuwacell, Product Name: M5). FIG. 5A shows representative cell morphology during iMSC derivation from engineered HGF-iPSCs. The typical MSC surface markers, CD73, CD90 and CD105, in WT-iPSCs and E-HGF-iMSCs (P2) were analyzed by flow cytometry (FIG. 5B). The HGF expression in WT-iPSCls, WT-iMSCs and E-HGF-iMSCs (P2) was analyzed by flow cytometry (FIG. 5C). The details for CD73/CD90/CD105 staining were as follows. Cells were dissociated and collected through TrypLE treatment. Cells were washed with 1 ml PBS and centrifuged at 300×g for 15-20 seconds. The supernatant was discarded and 50 μl/tube of Anti-CD73-APC Antibody (BD Pharmingen, #559869) or Anti-CD90-APC Antibody (BD Pharmingen, #560847) or Anti-CD105-APC Antibody (BioLegend, #800508) diluted in FACS buffer was added. The combination was mixed gently and incubated for 30 minutes at 4 °C. The cells were again washed in 1 ml of FACS buffer and then centrifuged at 300×g for 15-20 seconds. The supernatant was removed and 200 μl of FACS buffer added. The samples were then run on the flow cytometer.

As shown in FIGS. 5A and 5B, the E-HGF-iMSCs of P2 still had the typical phenotype of MSC, suggesting that the E-HGF-iMSCs could stably maintain the stem cell characteristic of MSC during the prolonged expansion. As shown in FIG. 5C, the expression level of HGF in the E-HGF-iMSCs is much higher than that of the WT-iMSCs.

### Example 7

### Experimental Procedure:

The WT-iPSCs and the E-HGF-iPSCs produced in Example 5 were cultured and expanded in ncEpic medium (Nuwacell) for 9 passages. The WT-iPSCls and the E-HGF-iPSCs at P1 and P10 were differentiated into iMSCs (hereinafter referred to as WT-iPSC-P1-iMSC-P0 cells, WT-iPSC-P10-iMSC-P0 cells, E-HGF-iPSC-P1-iMSC-P0 cells and E-HGF-iPSC-P10-iMSC-P0 cells, respectively) according to the protocol described in Examples 1, 3 and 4 of Patent CN110592007B, respectively. Then, the above differentiated iMSCs were separately cultured and expanded in an expansion medium (Nuwacell, Product Name: M5) for 4 or 7 passages. HGF expressions in WT-iPSC-P1-iMSC-P2 cells, WT-iPSC-P10-iMSC-P2 cells, E-HGF-iPSC-P1-iMSC-P2 cells and E-HGF-iPSC-P10-iMSC-P2 cells were analyzed by ELISA similar to Example 4 (FIG. 5D). Further, the HGF expressions in the WT-iPSC-P1-iMSC-P2 cells, WT-iPSC-P1-iMSC-P4 cells, E-HGF-iPSC-P1-iMSC-P2 cells, E-HGF-iPSC-P1-iMSC-P4 cells and E-HGF-iPSC-P1-iMSC-P7 cells were analyzed by ELISA similar to Example 4 (FIG. 5E).

As measured by ELISA, the level of the secreted HGF was about 1627 ng/mL/10⁶ cells for the E-HGF-iPSC-P1-iMSC-P2 cells, and it was about 1694 ng/mL/10⁶ cells for the E-HGF-iPSC-P1-iMSC-P4 cells. Further, FIG. 5D showed that the level of the secreted HGF for the E-HGF-iPSC-P1-iMSC-P2 cells is about 16.6 times as high as that of the WT-iPSC-P1-iMSC-P2 cells and the level of the secreted HGF for the E-HGF-iPSC-P10-iMSC-P2 cells is about 24.4 times as high as that of the WT-iPSC-P10-iMSC-P2 cells, and FIG. 5E showed that the level of the secreted HGF for the E-HGF-iPSC-P1-iMSC-P2 cells is about 16.6 times as high as that of the WT-iPSC-P1-iMSC-P2 cells, the level of the secreted HGF for the E-HGF-iPSC-P1-iMSC-P4 cells is about 17.5 times as high as that of the WT-iPSC-P1-iMSC-P4 cells, and the level of the secreted HGF for the E-HGF-iPSC-P1-iMSC-P7 cells is about 1.54 times as high as that of the E-HGF-iPSC-P1-iMSC-P2 cells. The above results suggested that the E-HGF-iMSCs exhibited high-level secretion of HGF, and could stably sustain the high-level secretion of HGF even after the E-HGF-iPSCs for iMSC differentiation had been subjected to the prolonged expansion up to P10. Moreover, the E-HGF-iMSC cells could be expanded stably for at lease 7 passages, with the high-level secretion of HGF maintained.

### Examples 8-11: Improved Efficacy of Engineered E-HGF-iMSCs in IPF Mice

### Example 8

### Experimental Procedure:

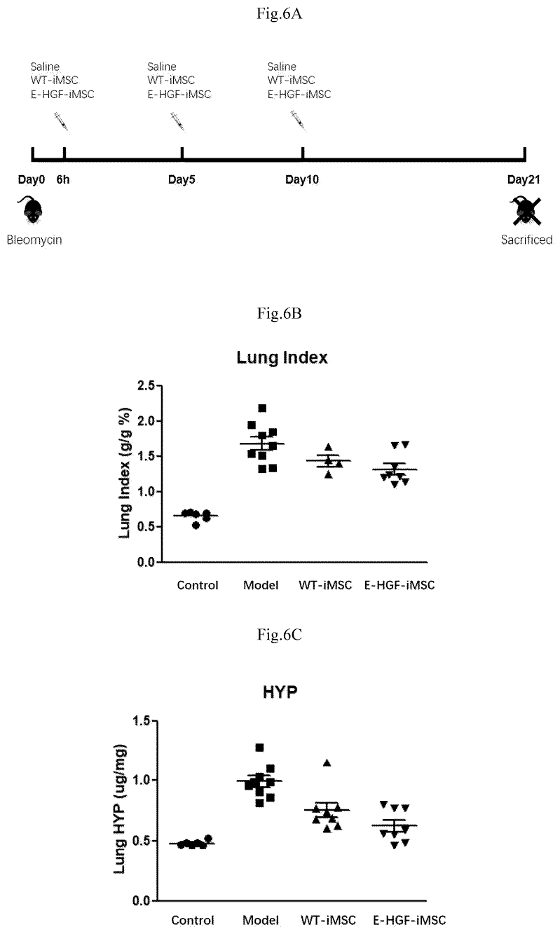

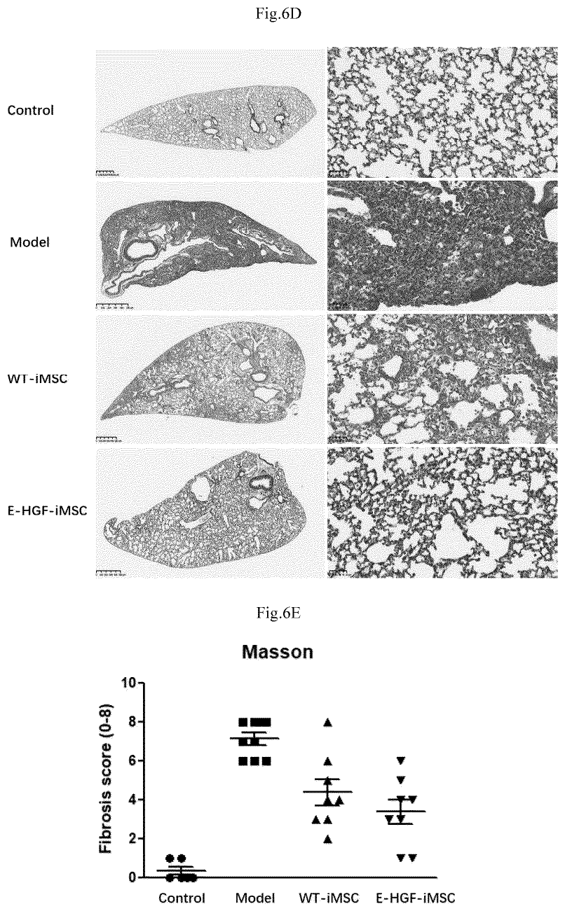

To induce IPF, C57BL/6N mice (Charles River) were anesthetized with 1% pentobarbital sodium (100 mg/mL). Then, 70 μg of Bleomycin dissolved in 50 μL saline was intratracheally administered to the mice. 1×10⁶ WT-iMSCs of P4 and E-HGF-iMSCs of P4 (Example 7) in 100 μL of saline were separately injected into the tail vein of the mice at 6 h, 5 days and 10 days after bleomycin administration (FIG. 6A). The mice injected with an equal volume of saline were referred to as the Model group, and the normal C57BL/6N mice were served as a blank control. All mice were sacrificed at Day 21. The body weights and lung weights of mice were measured, and the lung index was calculated as lung weights to body weights (FIG. 6B).

As shown in FIG. 6B, the lung indices for both the E-HGF-iMSC group and the WT-iMSC group were significantly reduced relative to that of the model group, and the E-HGF-iMSC group was superior to the WT-iMSC group and got closer to the normal group as control.

### Example 9

### Experimental Procedure:

The hydroxyproline (HYP) content was measured to assess the collagen deposition in lung tissue using an HYP detection kit (Nanjing Jiancheng Bioengineering Institute, Nanjing, China) according to the manufacturer's instructions. Specifically, lung tissue homogenates were hydrolyzed in 6M hydrochloric acid for 5 h at 95 °C, chloramine-T was subsequently added and pH was adjusted. Subsequently, after the addition of a color developer (dimethylaminobenzaldehyde), the samples were incubated for 15 min at 60 °C, and the absorbance at 550 nm was measured using a Microplate Reader (Bio-Rad, USA). HYP levels were determined by plotting a standard curve, and the results were presented as micro-grams per milligram in lung tissues (FIG. 6C).

As shown in FIG. 6C, the HYP contents for both the E-HGF-iMSC group and the WT-iMSC group were significantly reduced relative to that of the model group, and the E-HGF-iMSC group was superior to the WT-iMSC group and got closer to the normal group as control.

### Example 10

### Experimental Procedure:

For each mouse, the lung was fixed with 4% paraformaldehyde, embedded in paraffin. Sections were cut at 4 μm thicknesses and stained with hematoxylin and eosin (H&E). The experimental details were as follows. Deparaffinize the sections by two successive xylene baths for 10 min each. Hydrate the lung sections by passing through decreasing concentration of alcohol baths: 2 changes of absolute alcohol, 5 min each, 95% alcohol for 2 min and 70% alcohol for 2 min. Wash briefly in distilled water. Collect the sections on positive charged microscope slides. The slides were incubated with hematoxylin solution (Sigma) in a staining jar for 10 min to stain the nuclei. Transfer the slides to a staining jar with tap water till the water was clear. Then, transfer the slides to a staining jar with Eosin solution (Sigma) for 3 min. Successively transfer the slides into staining jar with 70% ethanol for 20 sec, 90% ethanol for 20 sec, 100% ethanol for 1 min and xylene for 3 min. Take out slides from xylene and place the slides in a fume hood till the slides were dry. Mount the slides with xylene-based mounting media and cover with cover slides. Clips were used to press the slides to squeeze bubbles. Store the slides at room temperature. At least 5 random fields of view of each group were imaged with a digital microscope camera.

The results were shown in FIG. 6D. The results showed that the collagen deposition was significantly alleviated in both the E-HGF-iMSC group and the WT-iMSC group than the model group, and the E-HGF-iMSC group was better than the WT-iMSC group and got closer to the normal group as control.

### Example 11

### Experimental Procedure:

For each mouse, the lung was fixed with 4% paraformaldehyde, embedded in paraffin. Sections were cut at 4 μm thicknesses and stained with a Masson staining kit (Nanjing Jiancheng Bioengineering Institute, Nanjing, China) according to the manufacturer's instructions. The experimental details were as follows. Lung section was deparaffinized and rehydrated as described in Example 10. The lung sections were washed with distilled water. The lung sections were mordant in preheated Bouin Fluid for either one hour at 56-60 °C or overnight at room temperature. Sections were then stained in Weigert's iron hematoxylin solution for 10 min. Sections were then washed with running tap water for 10 min and rinsed in distilled water. Sections were stained in Biebrich scarlet-acid fuchsin solution for 10-15 min and then rinsed in distilled water. Sections were then differentiated in phosphomolybdic-phosphotungstic acid solution for 10-15 min. Slide sections were examined watched with naked eyes and once collagen-rich areas lost red color and turned clear, proceeded to the next step. Slides were transferred directly to aniline blue solution and stained for 5-10 min. Slides were then rinsed in distilled water and differentiated in 1% acetic acid solution for 2-5 min. Slides were then washed in distilled water. Slides were subjected very quickly through 95% alcohol to dehydrate tissue followed by absolute alcohol to wipe off Biebrich scarlet-acid fuchsin staining and cleared in xylene. Slides were mounted with xylene-based mounting media and covered with cover slides. Any bubbles were removed and the slides stored at room temperature. At least 5 random fields of view of each group were imaged with a digital microscope camera. Each successive field was individually assessed for severity of interstitial fibrosis and allotted a score between 0 and 8 using a predetermined scale of severity (Table 3). The results are shown in FIG. 6E.

TABLE 3
Criteria for grading lung fibrosis:

| Grade of | |
|---|---|
| fibrosis | Histological features |
| 0 | Normal lung |
| 1 | Minimal fibrosis thickening of alveolar or bronchiolar walls |
| 2 | Moderate thickening of walls without obvious damage to lung |
| | architecture |
| 3 | Severe thickening of walls with obvious structure disorder |
| 4 | Severe thickening of walls with obvious damage to lung |
| | architecture |
| 5 | Increased fibrosis with definite damage to lung structure |
| | and formation of fibrous bands or small fibrous masses |
| 6 | Severe distortion of structure and large fibrous areas |
| 7 | “honeycomb lung” is placed in this category |
| 8 | Total fibrous obliteration of the field |

As shown in FIG. 6E, the fibrosis scores for both the E-HGF-iMSC group and the WT-iMSC group were significantly lower than that of the model group, and the E-HGF-iMSC group was lower than the WT-iMSC group and got closer to the normal group as control. Taking the results of FIGS. 6B-6E together, the E-HGF-iMSCs showed improved efficacy for IPF in contrast to the WT-iMSCs.

One skilled in the art would readily appreciate that the methods, compositions, and products described herein are representative of exemplary embodiments, and not intended as limitations on the scope of the disclosure. It will be readily apparent to one skilled in the art that varying substitutions and modifications may be made to the present disclosure disclosed herein without departing from the scope and spirit of the disclosure.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure is not to be limited in terms of the particular embodiments described in this application, which are intended as single illustrations of individual aspects of the disclosure. All the various embodiments of the present disclosure will not be described herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

1. A genetically modified pluripotent stem cell (PSC), comprising an expression cassette integrated at a target locus in the genome thereof, wherein the expression cassette comprises an exogenous polynucleotide encoding a hepatocyte growth factor (HGF).

2. The genetically modified PSC of claim 1, wherein the target locus is Rosa26.

3. The genetically modified PSC of claim 1, wherein the expression cassette comprises a promoter operably linked to the polynucleotide encoding the HGF, wherein the promoter is selected from the group consisting of EF1a, PGK, CAG, CMV, CLP, and UBC.

4. The genetically modified PSC of claim 3, wherein the expression cassette further comprises an anti-silencing ubiquitous chromatin opening element (UCOE) such as 1550F shown in SEQ ID NO: 1, and the UCOE is operably linked to the promoter.

5. The genetically modified PSC of claim 3, wherein the promoter is the EF1a promoter.

6. The genetically modified PSC of claim 4, wherein the expression cassette comprises the combination of the EF1a promoter with the UCOE.

7. The genetically modified PSC of claim 1, wherein the expression cassette further comprises a polynucleotide coding a signal peptide for directing the extracellular secretion of the HGF.

8. The genetically modified PSC of claim 7, wherein the signal peptide is shown in SEQ ID NO: 20.

9. A method of producing a genetically modified PSC, the method comprising:
introducing into PSC a first construct comprising a site-specific endonuclease capable of introducing a double strand break at a target locus in the genome of the PSC and a second construct comprising an expression cassette comprising an exogenous polynucleotide encoding a hepatocyte growth factor (HGF) and a pair of homology arms specific to the target locus and flanking the expression cassette such that the expression cassette in the second construct is integrated into the genome of the PSC at the target locus via homologous recombination, thereby obtaining the genetically modified PSC.

10. The method of claim 9, wherein the target locus is Rosa26.

11. The method of claim 9, wherein the expression cassette comprises a promoter operably linked to the polynucleotide encoding the HGF, wherein the promoter is selected from the group consisting of EF1a, PGK, CAG, CMV, CLP, and UBC.

12. The method of claim 11, wherein the expression cassette further comprises an anti-silencing ubiquitous chromatin opening element (UCOE) such as 1550F shown in SEQ ID NO: 1, and the UCOE is operably linked to the promoter.

13. The method of claim 11, wherein the promoter is the EF1a promoter.

14. The method of claim 12, wherein the expression cassette comprises the combination of the EF1a promoter with the UCOE.

15. The method of claim 9, wherein the expression cassette further comprises a polynucleotide coding a signal peptide for directing the extracellular secretion of the HGF.

16. The method of claim 15, wherein the signal peptide is shown in SEQ ID NO: 20.

17. The method of claim 9, further comprising continuously expanding the genetically modified PSC for multiple passages such as at least 4 or 9 passages.

18. A genetically modified induced mesenchymal stem cell (iMSC), comprising an expression cassette integrated at a target locus in the genome thereof, wherein the expression cassette comprises an exogenous polynucleotide encoding a hepatocyte growth factor (HGF).

19. The genetically modified iMSC of claim 18, wherein the target locus is Rosa26.

20. The genetically modified iMSC of claim 18, wherein the expression cassette comprises a promoter operably linked to the polynucleotide encoding the HGF, wherein the promoter is selected from the group consisting of EF1a, PGK, CAG, CMV, CLP, and UBC.

21. The genetically modified iMSC of claim 20, wherein the expression cassette further comprises an anti-silencing ubiquitous chromatin opening element (UCOE) such as 1550F shown in SEQ ID NO: 1, and the UCOE is operably linked to the promoter.

22. The genetically modified iMSC of claim 20, wherein the promoter is the EF1a promoter.

23. The genetically modified iMSC of claim 21, wherein the expression cassette comprises the combination of the EF1a promoter with the UCOE.

24. The genetically modified iMSC of claim 18, wherein the expression cassette further comprises a polynucleotide coding a signal peptide for directing the extracellular secretion of the HGF.

25. The genetically modified iMSC of claim 24, wherein the signal peptide is shown in SEQ ID NO: 20.

26. The genetically modified iMSC of claim 24, wherein the genetically modified iMSC secretes the HGF at a level of at least 16 times as high as that of wild type iMSC.

27. A method of producing a genetically modified iMSC, the method comprising:
providing the genetically modified PSC according to claim 1; and
differentiating the genetically modified PSC into iMSC, thereby producing the genetically modified iMSC.

28. The method of claim 27, further comprising continuously expanding the genetically modified iMSC for multiple passages such as at least 4 or 7 passages.

29. A method of producing a genetically modified iMSC, the method comprising:
introducing into iMSC a first construct comprising a site-specific endonuclease capable of introducing a double strand break at a target locus in the genome of the iMSC and a second construct comprising an expression cassette comprising an exogenous polynucleotide encoding a hepatocyte growth factor (HGF) and a pair of homology arms specific to the target locus and flanking the expression cassette such that the expression cassette in the second construct is integrated into the genome of the iMSC at the target locus via homologous recombination, thereby obtaining the genetically modified iMSC.

30. The method of claim 29, wherein the target locus is Rosa26.

31. The method of claim 29, wherein the expression cassette comprises a promoter operably linked to the polynucleotide encoding the HGF, wherein the promoter is selected from the group consisting of EF1a, PGK, CAG, CMV, CLP, and UBC.

32. The method of claim 31, wherein the expression cassette further comprises an anti-silencing ubiquitous chromatin opening element (UCOE) such as 1550F shown in SEQ ID NO: 1, and the UCOE is operably linked to the promoter.

33. The method of claim 31, wherein the promoter is the EF1a promoter.

34. The method of claim 32, wherein the expression cassette comprises the combination of the EF1a promoter with the UCOE.

35. The method of claim 29, wherein the expression cassette further comprises a polynucleotide coding a signal peptide for directing the extracellular secretion of the HGF.

36. The method of claim 35, wherein the signal peptide is shown in SEQ ID NO: 20.

37. The method of claim 29, further comprising continuously expanding the genetically modified iMSC for multiple passages such as at least 4 or 7 passages.

38. A pharmaceutical composition, comprising the genetically modified iMSC of claim 18 and a pharmaceutically acceptable carrier.

39. A method for treating or preventing idiopathic pulmonary fibrosis (IPF), comprising administering the genetically modified iMSC of claim 18 to a subject in need thereof.
